(19) United States
(12) Patent Application Publication
(10) Pub. No.: US 2019/0290629 A1
(43) Pub. Date: Sep. 26, 2019

(54) NIRAPARIB SUSTAINED AND CONTROLLED RELEASE PHARMACEUTICAL COMPOSITION AND USE THEREOF

(71) Applicants: Yong Gan, Shanghai (CN); Bingxue Meng, Shanghai (CN); Yan Liu, Shanghai (CN); Chunliu Zhu, Shanghai (CN); Shiyan Guo, Shanghai (CN)

(72) Inventors: Yong Gan, Shanghai (CN); Bingxue Meng, Shanghai (CN); Yan Liu, Shanghai (CN); Chunliu Zhu, Shanghai (CN); Shiyan Guo, Shanghai (CN)

(73) Assignee: SCIENCE RAINBOW BIOPHARMA CO. LTD, Suzhou City (CN)

(21) Appl. No.: 16/442,049

(22) Filed: Jun. 14, 2019

Related U.S. Application Data
(63) Continuation of application No. PCT/CN2017/116561, filed on Dec. 15, 2017.

(30) Foreign Application Priority Data
Dec. 16, 2016 (CN) 201611170036.X

(51) Int. Cl. A61K 31/4439

(52) U.S. Cl. CPC A61K 31/4439

(57) ABSTRACT

Provided are a niraparib sustained and controlled release pharmaceutical composition and use thereof. The sustained and controlled release pharmaceutical composition contains dissolution-improved niraparib and a matrix polymer used for regulating release rate; the steady-state plasma concentration trough value C_{min,ss} of the pharmaceutical composition is 0.5-4 μM; the steady-state plasma concentration peak value C_{max,ss} is 0.8-6 μM.

This patent application is a continuation of co-pending PCT/CN2017/116561, filed Dec. 15, 2017, which claims priority to Chinese Application No. 201611170036.X, filed Dec. 16, 2016, the entire teachings and disclosure of which are incorporated herein by reference thereto.

### TECHNICAL FIELD

The present invention belongs to biopharmaceutical field, and in particular relates to a niraparib sustained and controlled release pharmaceutical composition and the use thereof for preparing a drug for preventing or treating a tumor. The composition according to the present invention has a controlled release behavior, blood concentration in vivo and PARP enzyme inhibitory activity.

### BACKGROUND

Niraparib, chemical name: (S)-2-(4-(piperidin-3-yl)phenyl)-2H-indazole-7-carboxamide, the molecular formula: C₁₉H₂₀N₄O, the molecular weight: 320.39, has the following chemical structure:

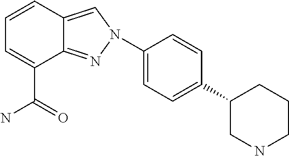 [SMILES: NC(=O)c1cccc2cn(-c3ccc([C@@H]4CCCNC4)cc3)nc12]

Niraparib (trade name Zejula) is a PARP enzyme inhibitor developed by the US biopharmaceutical company Tesaro Inc. and was approved by the US Food and Drug Administration (FDA) in December 2014, and Tesaro Inc. submitted with China CFDA in August 2017 a clinical application for indications of maintenance therapy in patients with recurrent epithelial ovarian, fallopian tube or primary peritoneal cancer, wherein these patients responded completely or partially to platinum-based chemotherapy.

Thousands of DNA damage occurs in each cell per day, and there are two types of DNA damage, single-strand breaks and double-strand breaks. PARP (polyadenosine diphosphate-ribose polymerase) mainly repairs single-strand breaks, and proteins encoded by BRCA1 and BRCA2 genes are involved in the repair of DNA double-strand damage through the homologous recombination (HR) pathway. In tumor cells, PARP inhibitors inhibit PARP activity, and single-strand DNA breaks in cells cannot be repaired and thus accumulated. Continuous single-strand DNA damage will be converted to double-stranded DNA damage during DNA replication, due to that BRCA1/2 Gene-deficient tumor cells cannot repair double-stranded DNA damage through HR, which will lead to the cessation of DNA replication fork, producing cytotoxicity, resulting in synthetic lethality, and finally killing tumor cells by targeting.

Poly ADP transferase (PARP) is a key factor in the DNA excision repair pathway, and niraparib can inhibit PRAP enzyme activity, making the broken single strands of DNA unrepairable, increasing genomic instability, and thus leading to cell apoptosis, and the drug has a strong killing effect especially for tumor cells with homologous recombination repair defects. This mechanism of niraparib makes it possible to treat two or more types of combined-type tumors; in addition, because niraparib has specific inhibition for damaged DNA repair pathway, the drug will also avoid tumor resistance after chemotherapy, enhance DNA damage, and improve the anti-tumor efficacy of previous chemotherapy drugs.

According to the data from the research on late ovarian cancer published by Tesaro and the reviews published by the FDA (FDA reviews, NDA 4074987), for the patients with ovarian cancer of BRCA gene mutation, after the first chemotherapy, if niraparib was orally administered once daily, the median “progression free survival time” was 21 months, vs. 5.5 months in the placebo control group; for patients without BRCA gene mutations, the median “progression free survival time” was 9.3 months, vs. 3.9 months in the placebo control group, and the differences were significant.

To date, the dose form of the new drug applied by Tesaro is an immediate release capsule preparation of niraparib tosylate monohydrate, 100 mg niraparib/capsule, and several clinical studies show that (FDA reviews, NDA 4074987), the absorption of niraparib is fast, oral bioavailability can reach 73%, blood concentration peak time is 3 hours, and plasma half-life is more than 30 hours, and its plasma exposure is not affected by food, and its exposure and the maximum blood concentration are multiplied with the increase of the dose. At present, the dose for clinical phase II/III 300 mg/time/day, and the steady-state blood concentration is reached on days 12-14, and the peak and trough values are about 4.4 μM and 2.0 μM, respectively.

However, the conventional oral immediate release capsule that are being studied still has some limitations, which are mainly manifested by dose-limiting toxicity, and the steady-state blood concentration peak value is several times or even ten times higher than the PARP enzyme IC90 value, resulting in more serious toxic and side effects, limiting the efficacy of the drug. The dose-limiting toxicity of niraparib is thrombocytopenia and anemia. In clinical studies, 69% of patients reduced dose or stopped taking the drug due to toxic and side effects, 15% of patients discontinued the treatment due to toxic and side effects, and 25% of patients had 3-4 grade anemia, 29% of patients had grade 3-4 platelet reduction, and 30% of patients had neutropenia, and severe toxic and side effects.

In order to further improve the clinical therapeutic effect of niraparib on tumors and reduce the toxic and side effects of the drug, it is necessary to provide an excellent preparation to prevent the peak blood concentration from being too high and accurately regulate the fluctuation range of blood concentration of niraparib. One object of the present invention is to develop a niraparib pharmaceutical composition, in which the releasing behavior of niraparib is controlled and the absorption rate and absorption time of niraparib in the gastrointestinal tract are accurately adjusted to prevent the blood concentration from rising sharply, and the blood concentration level in vivo of niraparib and its fluctuation range are adjusted to increase and maintain the blood concentration required for PARP enzyme inhibition in vivo, so as to further improve the anti-tumor effect of niraparib, and reduce the adverse reactions after administration. Another object of the present invention is to provide an excellent preparation that can minimize the size and/or amount of tablets or capsules required for a therapeutically effective dose, with as less administration times as possible, so as to improve patient compliance.

After searching, the inventor did not find patents related to niraparib preparation, and research on the oral sustained and controlled release preparation of niraparib. In order to further improve the clinical efficacy of niraparib and accurately control the blood concentration in vivo and the enzyme inhibition level, to reduce the after-administration adverse reactions in patients with tumor, and to improve patient compliance, the present invention discloses a niraparib pharmaceutical composition with controlled release behavior in vivo.

### SUMMARY OF THE PRESENT INVENTION

The large dose administration form of an immediate release capsule of niraparib often leads to high steady-state blood peak concentration after oral administration. The high peak value leads to many side effects, affecting the life quality of patients, meanwhile, the dose-limiting toxicity affects the efficacy of the drug.

According to the biological properties of niraparib and the efficacy and safety requirements for clinical treatment, in order to overcome the defects of the current preparation, the present invention provides a niraparib pharmaceutical composition whose in vivo absorption behavior, blood concentration and PARP enzyme inhibition level are controllable, to further improve the clinical efficacy of niraparib, reduce after-administration adverse reactions in patients with tumor, and improve patient compliance. The present invention relates to a novel drug composition with improved niraparib drug loading and/or oral absorption and/or bioavailability and/or blood concentration control and/or enzyme inhibition level control, and a use thereof for the treatment of cancer as a sole preparation or in combination with other therapies.

The niraparib sustained and controlled release pharmaceutical composition in the present invention has a controllable release behavior, and the release behavior and the release amount thereof can be controlled in a release medium in accordance with the sink condition within a predetermined period of time. When determining the release behavior in a buffer solution with a pH of 1.2-7.8 at 37° C. using the apparatus II of the dissolution test method in the Chinese Pharmacopoeia, the 1 hour release amount of niraparib is less than 50%, preferably 30%, more preferably 10-25% of the total amount of niraparib; the 16 hour release amount of niraparib is greater than 80%, more preferably 90% of the total amount of niraparib.

For the niraparib sustained and controlled release pharmaceutical composition in the present invention, the absorption rate and absorption time of niraparib in the gastrointestinal tract can be adjusted by controlling the release behavior and the release amount, and the controlled absorption behavior may further control the niraparib blood concentration level in vivo and fluctuation range thereof, thus maintaining the blood concentration in vivo stable for a long time, with a small fluctuation of blood concentration. The niraparib pharmaceutical composition in the present invention has an effective steady-state blood concentration trough value of 0.5 μM<C_{min,ss}<4 μM, or even 1 μM<C_{min,ss}<3 μM in the canine; and a steady-state blood concentration peak value of 0.8 μM<C_{max,ss}<6 μM, even 2 μM<C_{max,ss}<5 μM, and the steady-state blood concentration peak/trough ratio is preferably less than 2, more preferably less than 1.5.

Compared with the immediate release capsule under study, at the same dose of niraparib, the maximum blood concentration value (Cₘₐₓ) is reduced by at least 10%-50%, and the time for reaching the blood concentration peak (Tₘₐₓ) is prolonged by at least 50% (or even 200%-600%) for the pharmaceutical composition in the present invention. The steady-state blood concentration level of niraparib, the fluctuation range of free blood concentration, PARP enzyme inhibition, safety in vivo and administration times can be adjusted by controlling blood concentration, peak time and area under the concentration-time curve.

The niraparib sustained and controlled release composition in the present invention comprises a niraparib in an improved dissolution form and a matrix polymer for adjusting the drug release rate, depending on the dosage form, the composition may further comprise other additives, for example, one or a combination of two or more selected from pharmaceutical excipients, such as disintegrants, plasticizers, porogens, swelling materials, fillers, osmotic pressure regulators (also known as osmotic agents), lubricants, binders (also known as adhesives), colorants (also known as coloring agents), anti-adherents (also known as anti-adhesives), opacifiers, diluents, coating powders, semi-permeable controlled release coating film materials, seal coating materials, and/or other pharmaceutically acceptable additives and the like.

The active drug niraparib in the niraparib sustained and controlled release pharmaceutical composition in the present invention is a poorly soluble drug, and in order to achieve good absorption and oral bioavailability, the solubilization treatment can be firstly performed to the drug to obtain the niraparib in an improved dissolution form, thereby improving the dissolution of drug. Without being limited by any theory, the inventor believes that the solubilization treatment comprises preparing the drug into a niraparib salt, such as a hydrochloride, a phosphate, a besylate, a camphorate, a maleate, a sulfate, or the like; or mixing niraparib with a matrix polymer which can improve the solubility of the drug, such that the dispersion specific surface area of the active drug preparation in composition powder is changed, thereby improving the dissolution property of the drug. The solubilization treatment may comprise co-grinding, high pressure homogenization, coprecipitation, solvent evaporation or melt extrusion, etc. In the specification of the present invention, unless otherwise specified, in the specially described terms such as niraparib hydrochloride, niraparib maleate, etc., said “niraparib” means niraparib free base.

The niraparib in an improved dissolution form in the present invention comprises: a corresponding salt compound of niraparib free base, a co-grinding mixture prepared with niraparib and other matrix excipients, a niraparib nanocrystal or a niraparib solid dispersion or the like; wherein the compound in the salt form is a pharmaceutically acceptable salt thereof, which may be selected from the group consisting of hydrochloride, phosphate, besylate, maleate, sulfate, and d-camphorate or the like. The niraparib in a salt form can significantly improve its solubility in water, the raw drug of the niraparib in a salt form can be directly used for the preparation of the sustained and controlled release preparation; and the niraparib co-grinding mixture, the nanocrystal or the solid dispersion consists essentially of the active drug niraparib and a pharmaceutically acceptable matrix polymer for improving solubility and optionally other additives such as plasticizers and the like.

The niraparib co-grinding mixture in the present invention consists of the active drug niraparib, a matrix polymer for improving solubility, and optionally pharmaceutically acceptable other additives, and it is prepared by mixing and co-grinding the above ingredients. The particle size of the sufficiently grinding drug powder can generally be less than 100 microns. Without being limited by any theory, the co-grinding can increase the dispersion specific surface area of the drug in the solid preparation powder, thereby improving the dissolution property of the drug.

According to the present invention, in the co-grinding mixture, based on the total weight of the co-grinding mixture, the weight percentage of niraparib is 5-60 wt %, preferably 20-40 wt %, and the weight percentage of the matrix polymer for solubilization is 40-95 wt %, preferably 40-80 wt %, the weight percentage of the other additives is from 0-15 wt %, preferably from 0.2-10 wt %. The total amount of the above components is 100 wt %.

For the range of the components of the present invention, for example, for the range of the respective ingredients in the above co-grinding mixture, it is understood that any value between the lower limit values and any value between the upper limit values are within the scope of the present invention, for example, the weight percentage of niraparib is 5-60 wt %, preferably 20-40 wt %, and it is understood as that the lower limit includes any number in the range of 5-20%, such as 6%, 7%, 8%, 9%, 10%, 11%, 12%, 13%, 14%, 15%, 16%, 17%, 18%, 19%; and the upper limit includes any number in the range of 40-60%, such as 41%, 42%, 43%, 44%, 45%, 46%, 47%, 48%, 49%, 50%, 51%, 52%, 53%, 54%, 55%, 56%, 57%, 58% 59%. For the weight percentage of the matrix polymer for solubilization, similar to the above, the weight percentage thereof is 40-95 wt %, preferably 40-80 wt %, which can be understood as any range within the ranges of 40%-(80%-95%), and the detailed description thereof is omitted.

The niraparib nanocrystal in the present invention consists of the active drug niraparib, a matrix polymer, and optionally other additives, and it is prepared by subjecting the components to high-pressure homogenization or coprecipitation method. The high-pressure homogenization method comprises the following steps: the crude crystal suspension prepared by the high-speed shearing of the aqueous solution of the active drug niraparib and the matrix polymer is fed to a high-pressure homogenizer, and high-pressure homogenized for several times until the crystal particles of 1000 nm or less are prepared, which is then lyophilized to give a uniformly dispersed niraparib nanocrystal powder. The coprecipitation method comprises the following steps: the active drug niraparib is first dissolved in a small amount of an organic solvent such as acetone, and quickly added to a large amount of an aqueous solution in which the matrix polymer is dissolved, and which is then treated by high frequency ultrasound using ultrasonic probe to ensure the formation of the active drug crystal nucleus and uniform dispersion, until a stably dispersed nanocrystal solution is formed, and then the solution is lyophilized to give a uniformly dispersed niraparib nanocrystal powder. By preparing the nanocrystals, the particle diameter of the active drug niraparib dispersed in the solid powder can be reduced, and the specific surface area of the active drug is remarkably increased, such that the dissolution property of the drug is improved.

In the niraparib nanocrystal, based on the total weight of the niraparib nanocrystal, the weight percentage of niraparib is 10-100 wt %, preferably 20-50 wt %; the weight percentage of the matrix polymer for solubilization is 0-75%, preferably 0-65%, and the weight percentage of other additives is 0-10 wt %, preferably 0-5 wt %. The total amount of the above components is 100 wt %. The nanocrystalline composition has a particle size of 50-1000 nm. For the respective ranges of the above components, similar to the understanding of the above-described co-grinding mixture, any value between the lower limit values and any value between the upper limit values are within the scope of the present invention, the detailed description thereof is omitted.

The solid dispersion in the present invention consists of active drug niraparib, a matrix polymer for solubilization, and other additives. In the solid dispersion, based on the total weight of the solid dispersion, the weight percentage of niraparib is 5-50 wt %, preferably 10-40 wt %, more preferably 20-40 wt %, and the weight percentage of the matrix polymer for solubilization is 45-95 wt %, preferably 50-80 wt %, and the weight percentage of other additives (such as colloidal silicon dioxide, polyethylene glycol stearate, etc.) is 0-12 wt %, preferably 0-10 wt %. The total amount of the above components is 100 wt %. For the respective ranges of the above components, similar to the understanding of the above-described co-grinding mixture, any value between the lower limit values and any value between the upper limit values are within the scope of the present invention, the detailed description thereof is omitted. The solid dispersion composition can be produced by a solvent evaporation method or a melt extrusion method. The solvent evaporation method is carried out by dissolving the drug niraparib, the matrix polymer and/or other additives in a volatile organic solvent or an organic mixed solvent, evaporating the organic solvent under reduced pressure, and transferring the intermediate product obtained by evaporating the organic solvent to a vacuum oven for drying to produce a niraparib solid dispersion. The melt extrusion method is carried out by: directly and slowly adding the drug niraparib, the matrix polymer and optionally other additive powders, which have been well mixed, to the melt extruder, and collecting the melt extrudate. Without being limited by any theory, the solid dispersion enables the active drug niraparib to exhibit a high energy-state solid dispersion state, and makes the drug dispersed in the solid powder of the preparation composition in a molecular form, such that the specific surface area of the drug is maximized, and the dissolution property of the drug is improved.

In the niraparib co-grinding mixture, niraparib nanocrystal and niraparib solid dispersion of the present invention, the active drug niraparib comprises niraparib free base and a pharmaceutically acceptable salt thereof, and the pharmaceutically acceptable salt may be selected from the group consisting of hydrochloride, phosphate, besylate, camphorate, maleate, sulfate, and the like.

In the niraparib co-grinding mixture, niraparib nanocrystal and niraparib solid dispersion of the present invention, the matrix polymer for solubilization means a polymer that can stabilize and/or solubilize niraparib particles or molecules, and it may be one or a combination of two or more selected from the group consisting of povidone, copovidone, polyoxyethylene, Soluplus, hypromellose phthalate (HPMCP), hydroxypropylcellulose acetate succinate, polyethylene glycol, poloxamer, polymethacrylic acid, polyethyl acrylate, 2-hydroxypropyl-β-cyclodextrin, hypromellose (HPMC), polymethacrylate, hydroxypropyl cellulose, cellulose acetate phthalate (CAP) and other common pharmaceutical polymeric excipients for solubilization; the other additives may be selected from pharmaceutically common plasticizers and/or lubricants, etc., the plasticizer may be one or a combination of two or more selected from PEG 4000, phthalates, small molecule surfactants such as Cremphor RH40 and polyoxyethylene (40) stearate and other pharmaceutically common plasticizers, the lubricant may be one or a combination of two or more selected from the common lubricants such as colloidal silicon dioxide, magnesium stearate, and the like.

The matrix polymer for adjusting release rate (hereinafter sometimes referred as a release regulator) in the present invention may be a sustained release matrix material well known to those skilled in the art, and it may be selected from cellulose derivatives, starch or derivatives thereof, alginate, acrylic acid or methacrylic acid derivatives, polyethylene oxide, gums and carbohydrate-based polymers, for example, it may be one or a combination of two or more selected from the group consisting of polyoxyethylene, hydroxypropyl cellulose, hypromellose, methyl cellulose, hydroxyethyl cellulose, ethyl cellulose, sodium alginate, povidone, copovidone, acrylic resin, and carbomer, preferably one or a combination of two or more selected from polyoxyethylene, hydroxypropyl cellulose, sodium alginate, hypromellose, and carbomer.

The niraparib sustained and controlled release pharmaceutical composition in the present invention comprises 50-900 parts by weight, preferably 80-700 parts by weight, more preferably 120-600 parts by weight, of the niraparib in an improved dissolution form; and 10-300 parts by weight, preferably, 20-250 parts by weight, more preferably 50-180 parts by weight, of the matrix polymer for adjusting release rate. More specifically, for the niraparib in an improved dissolution form, the niraparib oral sustained and controlled release pharmaceutical composition in the present invention comprises, 50-700 parts by weight of a compound of the niraparib in a salt form, and 10-300 parts by weight of a matrix polymer for adjusting release rate; or 50-700 parts by weight of a niraparib co-grinding mixture, and 10-200 parts by weight of a matrix polymer for adjusting release rate; or 50-800 parts by weight of a niraparib nanocrystal, and 0.1-250 parts by weight of a matrix polymer for adjusting release rate; or 50-900 parts by weight of a niraparib solid dispersion, and 20-300 parts by weight of a matrix polymer for adjusting release rate.

The niraparib pharmaceutical composition in the present invention may be a sustained and controlled release preparation containing a single sustained release phase, or an immediate and sustained double-effect release preparation containing both an immediate release phase and a sustained release phase.

The sustained release phase is a controlled release composition containing a pharmaceutically active ingredient. The controlled release phase is preferably selected from, but not limited to, a controlled release composition in a controlled release tablet, a controlled release pellet or a tablet; a controlled release composition in a tablet or a pellet core; a controlled release layer composition incorporated into a double-layer tablet, and any combinations thereof.

The immediate release phase is an immediate release composition containing a pharmaceutically active ingredient. The immediate release phase is preferably selected from, but not limited to, an immediate release composition in an immediate release tablet, an immediate release pellet or a tablet; and an immediate release coat layer coating on a controlled release tablet or a pellet core, and an immediate release layer composition in a double-layer controlled release tablet, and any combination thereof.

The immediate and sustained double-effect controlled release preparation comprises both a sustained release phase and an immediate release phase. In the immediate and sustained double-effect controlled release preparation, the pharmaceutically active ingredient in the immediate release phase accounts for 10-50 wt %, preferably 20-40 wt % of the total amount of the pharmaceutically active ingredient; the pharmaceutically active ingredient in the sustained release phase accounts for 50-90 wt %, preferably from 60-80 wt % of the total amount of the pharmaceutically active ingredient.

The niraparib pharmaceutical composition in the present invention can be prepared into the following dosage forms, including dosage forms such as a single sustained and controlled release preparation and/or an immediate and sustained double-effect release preparation, which are selected from the group consisting of sustained release microspheres, immediate and sustained double-effect release microspheres, single layer osmotic pump controlled release tablet, double-layer osmotic pump controlled release tablets, immediate and sustained double-effect release osmotic pump tablets, sustained release matrix tablets, immediate and sustained double-effect release matrix tablets, sustained release capsules and immediate and sustained double-effect release capsules or the like. Each unit of preparation in the dosage form (such as a single preparation or capsule) may contain 20 mg to 400 mg, preferably 50 mg to 400 mg of the pharmaceutically active ingredient, and the recommended total dose/day by human is 100-800 mg/day, preferably 200-500 mg/day, such that the blood concentration level in vivo may be maintained in an effective range required for the inhibition of PARP enzyme. The composition can improve the PARP enzyme inhibition effect and tumor treatment effect of niraparib, and reduce the toxic and side effects of the drug.

The present invention provides a use of the niraparib pharmaceutical composition in the preparation of a drug for preventing or treating a tumor, in particular a tumor selected from ovarian cancer, breast cancer, gastric cancer, lung cancer, blood cancer, pancreatic cancer, glioblastoma, epithelial ovarian cancer, metastatic brain cancer and the like.

The niraparib pharmaceutical composition provided by the present invention may be used in clinical treatment of various types of tumors, and the present invention does not exclude the combinations with other anti-tumor drugs.

Compared with common immediate release preparations, the present niraparib pharmaceutical composition has the following advantages:

1) blood concentration and the fluctuation range can be controlled, the safety window is broader, and the dosage and dosage regimen can be flexibly adjusted during clinical treatment, the dosage can be further increased to provide long-term and more effective inhibition of PARP enzyme activity and improved efficacy.

2) the drug absorption rate can be controlled, and the blood concentration range can be adjusted, the fluctuation of blood concentration is small, and the adverse reactions in the patient after administration are reduced;

3) the size and/or amount of tablets or capsules required for effective therapeutic doses is minimized, patient compliance is improved, at the same time, production, storage and transportation are facilitated, and thus commercial value is increased.

### BRIEF DESCRIPTION OF THE DRAWINGS

FIG. 1 is a schematic view showing the structure of an osmotic pump-type controlled release tablet;

FIG. 2 is a schematic view showing the structure of an osmotic pump-type immediate and sustained double-effect release tablet;

FIG. 3 is a schematic view showing the structure of a matrix-type immediate and sustained double-effect release double-layer tablet;

FIG. 4 is a schematic view showing the structure of a matrix-type immediate and sustained double-effect release coated tablet;

FIG. 5 is a schematic view showing the structure of a capsule containing an immediate release tablet and sustained release tablet;

FIG. 6 is a schematic view showing the structure of a capsule containing an immediate release pellet and a matrix-type sustained release pellet according to an embodiment of the present invention;

FIG. 7 is a schematic view showing the structure of a capsule containing a sustained release pellet coated with an immediate release coat according to an embodiment of the present invention;

FIG. 8 shows the release curve of the immediate and sustained double-effect release matrix tablet of Example 1;

FIG. 9 shows the release curve of the double-layer osmotic pump controlled release tablet of Example 3 in the release medium of pH 1.2, 4.5 and 6.8;

FIG. 10 shows the release curves of the sustained and controlled release preparations of Example 4, Example 5, Example 6, Example 7, Example 8, Example 9, and Example 10;

FIG. 11 shows a dissolution profile of the immediate release capsule of Comparative Example 1.

FIG. 12 shows a dissolution profile of the immediate release capsule of Comparative Example 2;

FIG. 13 shows a diagram showing the in vivo results of the immediate release capsule of Comparative Example 1 and the immediate and sustained double-effect matrix tablet of Example 1.

FIG. 14 shows the in vivo drug-time curves of the immediate release capsule of Comparative Example 1 and the double-layer osmotic pump controlled release tablet of Example 3;

FIG. 15 shows the in vivo drug-time curves of the immediate release capsule of Comparative example 2 and the immediate and sustained double-release double-layer osmotic pump controlled release tablet of Example 4; and

FIG. 16 shows the enzyme inhibition rate vs. time curve of the immediate release capsule of Comparative Example 2 and the immediate and sustained double-release double-layer osmotic pump controlled release tablet of Example 4 in PBMC.

### DETAILED DESCRIPTION OF THE EMBODIMENTS

In order to better illustrate the properties of the niraparib pharmaceutical composition in the present invention, the detailed descriptions of the present invention are provided hereinafter, however, these descriptions are not intended to limit the scope of the present invention:

The sustained and controlled release tablet, which is one of the types of niraparib compositions in the present invention, may be selected from the group consisting of an osmotic pump-type controlled release tablet, a matrix-type controlled release tablet and a sustained release pellet-based sustained and controlled release tablet; in which the osmotic pump-type controlled release tablet includes osmotic pump controlled release tablet and osmotic pump immediate and sustained double-effect release tablet, and the matrix-type controlled release tablet includes matrix-type sustained release tablet, matrix-type immediate and sustained double-effect double-layer tablet and matrix-type immediate and sustained double-effect coated tablet and the like, the sustained release pellet-based sustained and controlled release tablet includes sustained release pellet-based sustained release tablet and sustained release pellet-based immediate and sustained double-effect release tablet, and for the above-mentioned sustained and controlled release tablets, the release behavior described in the present invention can be achieved by the following embodiments.

1. Osmotic Pump-Type Controlled Release Tablet

The osmotic pump controlled release tablet in the present invention may be a single layer osmotic pump tablet, a single layer osmotic pump immediate and sustained double-effect release tablet, a double-layer osmotic pump controlled release tablet or a double-layer osmotic pump immediate and sustained double-effect release tablet.

The double-layer osmotic pump controlled release tablet in the present invention mainly comprises:

1) a controlled release drug layer made of a controlled release drug-containing layer composition, located in a rigid film shell, adjacent to the drug release hole;

2) a push layer (also referred to as a boost layer) made of the push layer composition, located in the rigid film shell, away from the side of the drug release hole;

3) an optional seal coating layer, sandwiched between the inner surface of the rigid film shell and the tablet core composed of the drug layer and the push layer, and it is obtained by drying the seal coating composition;

4) a rigid film shell with moisture permeability, which is obtained by drying a controlled release coating solution, and the film shell comprises one or more drug releasing holes at one end;

5) an optional, non-limiting aesthetic coat;

6) an optional, non-limiting immediate release drug-containing layer made of the immediate release drug-containing layer composition, located outside of the rigid film shell or optional aesthetic coat.

Wherein, niraparib accounts for 3-50 wt % of the total weight of the osmotic pump-type controlled release tablets.

The controlled release drug-containing layer composition comprises: 50-600 parts by weight, preferably 80-500 parts by weight, more preferably 120-400 parts by weight of niraparib in an improved dissolution form; 10-150 parts by weight, preferably 20-120 parts by weight, more preferably 30-100 parts by weight of the release regulator, and 0-40 parts by weight, preferably 0-30 parts by weight of the other common pharmaceutical excipients.

The niraparib in an improved dissolution form may be selected from the above-described niraparib salt, niraparib co-grinding mixture, nanocrystalline or solid dispersion. The release regulator may be one or a combination of two or more selected from povidone, copovidone, polyethylene oxide, carbomer, hypromellose, croscarmellose sodium, hydroxypropyl cellulose, and sodium dodecyl sulfate.

Other common pharmaceutical excipients in the controlled release drug-containing layer composition are, without limitation, selected from the group consisting of penetration promoter, lubricants, and colorants commonly used in pharmaceutical tablets, and the amounts thereof are conventional selection in the art. The penetration promoter is one or a combination of two or more selected from the group consisting of sodium chloride, lactose, mannitol, glucose, sucrose, and fructose, preferably sodium chloride, the amount thereof may be 0-20 parts by weight. The lubricant is one or a combination of two or more selected from the group consisting of sodium stearyl fumarate, magnesium stearate, colloidal silicon dioxide, talc, polyethylene glycols, and magnesium lauryl sulfate, the amount thereof may be 0-20 parts by weight. The colorant is one or a combination of two or more selected from the group consisting of iron oxide red, iron oxide yellow, iron oxide violet, iron oxide black, and the like, and the amount thereof may be 0-10 parts by weight.

The push layer composition typically comprises a permeation-promoting polymer for adjusting release rate, an osmotic pressure promoter, and other excipients.

The permeation-promoting polymer is a high molecular polymer which swells in an aqueous medium by absorbing water and promotes release of the drug from the drug layer. The permeation-promoting polymer for adjusting release rate may be a material well known to those skilled in the art, and comprises one or a combination of two or more selected from the group consisting of polyoxyethylene, hydroxypropylmethylcellulose, hydroxypropylcellulose, croscarmellose sodium, crospovidone, sodium carboxymethyl starch, low-substituted hydroxypropyl cellulose, hydroxypropyl methylcellulose, hydroxypropyl cellulose, croscarmellose sodium, crospovidone, copovidone, carbomer, alginic acid and/or a derivative thereof, and the amount thereof may be 10-300 parts by weight, preferably 20-250 parts by weight, more preferably 50-180 parts by weight.

The osmotic pressure promoter is one or a combination of two or more selected from the group consisting of sodium chloride, lactose, mannitol, glucose, sucrose, and fructose, preferably sodium chloride, and the amount thereof may be 20-150 parts by weight, preferably 25-100 parts by weight.

The other excipients in the push layer composition include, without limitation, a lubricant, a colorant, and the like, and the amount thereof may be 0.5-30 parts by weight, preferably 2-20 parts by weight. The lubricant is one or a combination of two or more selected from the group consisting of sodium stearyl fumarate and sodium stearate, and the amount thereof may be 0.2-15 parts by weight. The colorant is one or a combination of two or more selected from the group consisting of iron oxide black, iron oxide red, and iron oxide yellow, and the amount thereof may be 0.5-15 parts by weight.

The controlled release drug-containing layer constitutes a tablet core of the osmotic pump controlled release tablet together with the push layer. The controlled release drug-containing layer accounts for 40-80 wt % and the push layer accounts for 20-60 wt % based on the total weight of the tablet core.

The seal coating layer can be formed by spraying the seal coating solution onto the tablet core and drying the same. The seal coating solution generally comprises a seal coating material and a solvent. The seal coating material is one or a combination of two or more selected from the group consisting of hydroxypropylmethylcellulose, povidone, copovidone, hydroxyethylcellulose, hydroxypropylcellulose, polyethylene glycol, and stearic acid, but not limited thereto. The solvent includes one or a combination of two or more of ethanol, water, acetone, isopropyl alcohol, but not limited thereto. The thickness of the seal coating can affect the release of the pharmaceutical preparation and the amount thereof can be controlled by the amount sprayed, and generally, with a coating level of 0-10 wt % relative to the tablet core.

The rigid film shell may also be referred to as a controlled release coat layer, which is formed by spraying a controlled release coating solution onto a tablet core formed by a drug-containing layer and a push layer and drying the same, and the rigid film shell gains the weight by 3-20 wt %, preferably 5-15 wt % relative to the tablet core.

The controlled release coating solution comprises 4-40 parts by weight, preferably 10-30 parts by weight, of a semipermeable controlled release coating material, 0-20 parts by weight of a plasticizer, 0-20 parts by weight of a porogen, and 50-1000 parts by weight, preferably 200-800 parts by weight of a solvent.

The semipermeable controlled release coating material is one or a combination of two or more selected from the group consisting of cellulose acetate, ethyl cellulose, and acrylic resin.

The plasticizer is one or a combination of two or more selected from the group consisting of methyl phthalate, ethyl phthalate, dibutyl sebacate, triethyl citrate, tributyl citrate, tributyl acetocitrate, triacetin and castor oil.

The porogen is one or a combination of two or more selected from the group consisting of glycerin, povidone, copovidone, propylene glycol, polyethylene glycol, and a water-soluble inorganic salt.

The solvent is one or a combination of two or more selected from the group consisting of acetone, water, ethanol, isopropanol, dichloromethane, and methanol.

The film shell contains one or more drug release holes, and the drug release holes can be prepared by mechanical drilling or laser drilling. The drug release holes can have any geometric shapes, such as a circle, an ellipse, a square, a triangle, etc., with an average pore size ranging from 0.3-1.2 mm.

The aesthetic coat is formed by spraying the aesthetic coat solution onto the tablet core and drying the same, and a layer of aesthetic coat, without any limitations, may be applied, which is generally unrestrictedly coated onto a conventional double-layer osmotic pump tablet. For the immediate and sustained double-effect osmotic pump tablet with the immediate release phase coating, the aesthetic coat is rarely applied thereto. The aesthetic coat can improve the appearance of the preparation to improve the patient compliance and provide color identification. The aesthetic coat solution can be conventionally selected in the art, including Opadry known to those skilled in the art and other coating powders that can form the aesthetic coat. Further, the aesthetic coat solution may further include one or more selected from the group consisting of a colorant, a plasticizer, an opacifier, an anti-adhesive agent, and a solvent. The aesthetic coat typically gains the weight by 0-10 wt % relative to the tablet core.

The single layer osmotic pump controlled release tablet of the present invention mainly comprises a single layer tablet core and a controlled release coating film with release holes, wherein the single layer osmotic pump controlled release tablet may be prepared by a method comprising the following steps: uniformly mixing a formula dosage of the niraparib in an improved dissolution form, release regulator, osmotic pressure promoter and other common pharmaceutical excipients, granulating the same, and pressing a single layer tablet core; coating the controlled release coating film material onto the tablet core by using a suspension coating method which is well known to those skilled in the art; punching the same by using a laser drilling machine to form the single layer osmotic pump controlled release tablet. The niraparib in an improved dissolution form, the release regulator and the osmotic pressure promoter are the same as those described for the double-layer osmotic pump tablet. Other pharmaceutical excipients include permeation-promoting polymer, controlled release coating film, lubricant, colorant, and the like, same as the above described for the double-layer osmotic pump tablet. In the single layer osmotic pump controlled release tablet, based on the total weight of the single layer tablet core, the single layer tablet core comprises 50-700 parts by weight, preferably 80-600 parts by weight, more preferably 120-400 parts by weight of the niraparib in an improved dissolution form; 10-150 parts by weight, preferably 20-120 parts by weight, more preferably 30-100 parts by weight of the release regulator; and 1-400 parts by weight, preferably 1-300 parts by weight of other common pharmaceutical excipients. The amount of the porogen in the sustained release coating film is from 0-30 wt % based on the total weight of the sustained release coating film. Based on the total weight of the single layer osmotic pump controlled release tablet, the controlled release coating film gains the weight by 3-30 wt % relative to the single layer osmotic pump controlled release tablet.

When there is an immediate release drug-containing layer, the osmotic pump controlled release tablet is an immediate and sustained double-effect release osmotic pump tablet. The immediate release drug-containing layer can be prepared by spraying the immediate release drug-containing layer composition onto the tablet core and drying the same. The immediate release drug-containing layer composition comprises: 10-80 parts by weight, preferably 20-50 parts by weight, of the active ingredient niraparib, 0-100 parts by weight, preferably 0-100 parts by weight, of the matrix polymer component for solubilization, 0-30 parts by weight of other common pharmaceutical excipients and 100-2000 parts by weight of solvent. The matrix polymer component for solubilization is one or a combination of two or more selected from the group consisting of povidone, copovidone, Soluplus, hypromellose phthalate (HPMCP), polyethylene glycol, poloxamer, polymethacrylic acid, polyethyl acrylate, hypromellose (HPMC), polymethacrylate, and hydroxypropyl cellulose. The other common pharmaceutical excipients include additives which are commonly used in immediate release tablets and well known to those skilled in the art, such as crospovidone, microcrystalline cellulose, pharmaceutically acceptable surfactants (for example, sodium dodecyl sulfate), and the like; The solvent includes one or a combination of two or more of ethanol, acetone, and water.

For the immediate and sustained double-effect release osmotic pump tablet, the amount of the niraparib in the immediate release drug-containing layer is about 10-40 wt % of the total weight of the niraparib in the entire immediate and sustained double-effect release osmotic pump tablet, and the amount of the niraparib in the controlled release drug-containing layer is about 60-90 wt % of the total weight of the niraparib in the entire immediate and sustained double-effect release osmotic pump tablet.

The preparation method of the niraparib osmotic pump controlled release tablet comprises the following steps: \textcircled{1} preparation of niraparib in an improved dissolution form; \textcircled{2} preparation of the drug-containing layer; \textcircled{3} preparation of the optional push layer; \textcircled{4} preparation of the tablet cores; \textcircled{5} preparation of the seal coating; \textcircled{6} preparation of controlled release coating film; \textcircled{7} punching of the osmotic pump tablet controlled release coating film; \textcircled{8} preparation of the optional esthetic coat; \textcircled{9} preparation of the optional immediate release drug-containing layer. The above steps \textcircled{2}-\textcircled{9} can be carried out by a conventional tableting and coating method well known to those skilled in the art.

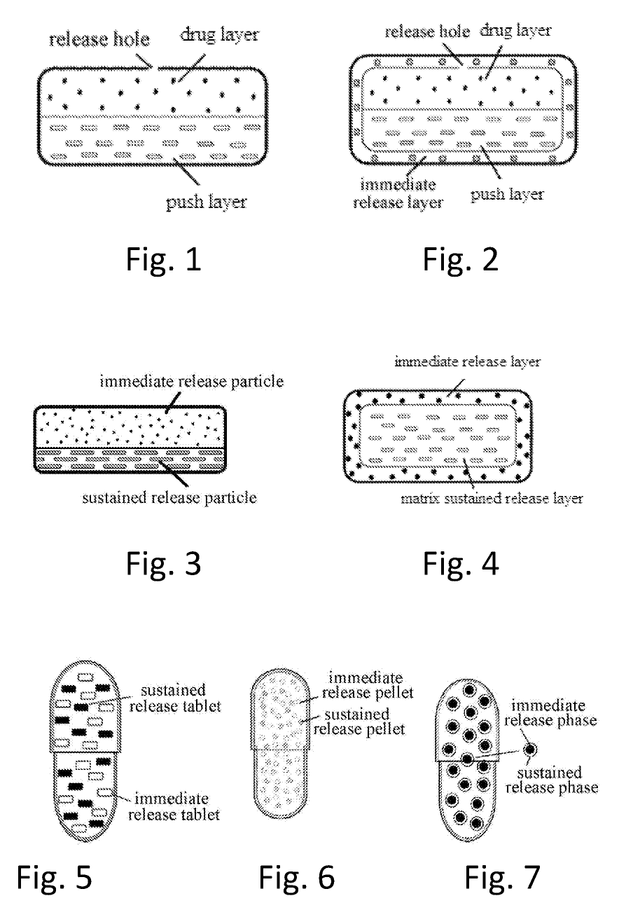

A tablet with a rigid film shell coated by an immediate release drug-containing layer is an osmotic pump immediate and sustained double-effect release tablet, and a tablet with a rigid film shell without coated by the immediate release drug-containing layer is a common osmotic pump controlled release tablet. FIG. 1 is a schematic view showing the structure of an osmotic pump-type controlled release tablet according to an embodiment of the present invention, and FIG. 2 is a schematic view showing the structure of an osmotic pump immediate and sustained double-effect release tablet according to an embodiment of the present invention.

The efficacy of niraparib can be improved by designing immediate and sustained double-effect release tablet, since the design of the immediate release phase ensures the rapid release of the drug at the earlier stage, and enables the drug to quickly reach the blood concentration level required for the effective PARP enzyme inhibition and thus exhibiting fast efficacy, while the design of the sustained release phase can ensure the steady release of the active ingredients at the later stage, ensuring the long-term maintenance of the blood concentration required for effective enzyme inhibition, thereby maintaining the inhibition of enzyme activity, improving the therapeutic effect, and reducing the toxic and side effects caused by large blood concentration fluctuations.

2. Matrix-Type Sustained and Controlled Release Tablets

The present invention provides a niraparib sustained release matrix tablet and/or a matrix tablet having a double-effect release behavior depending on the specifications of the drug and the requirement for treatment. The matrix-type controlled release tablet in the present invention is mainly composed of \textcircled{1} a sustained release phase (sustained release layer) containing a matrix polymer for adjusting release rate; and \textcircled{2} an optional immediate release phase (immediate release layer).

FIG. 3 is a schematic view showing the structure of a matrix-type immediate and sustained double-effect release double-layer tablet; and FIG. 4 is a schematic view showing the structure of a matrix-type immediate and sustained double-effect release coated tablet. The single layer tablet composed only of a sustained release phase containing a matrix polymer for adjusting release rate is a common sustained release matrix tablet, and the matrix tablet composed of a sustained release phase containing a matrix polymer for adjusting release rate and an immediate-release phase is an immediate and sustained double-effect release matrix tablet, and the immediate release layer and the sustained release layer can be stacked in the immediate and sustained double-effect release matrix tablet, or the immediate release layer may be also coated onto the sustained release layer. The design of the immediate release phase in the immediate and sustained double-effect release matrix tablet can well ensure the rapid release of the drug at the earlier stage, such that the drug quickly takes effect and reaches the therapeutic concentration, while the sustained release phase can ensure the steady release of the active ingredient at the later stage, such that the blood concentration maintains an effective level for a long time, thereby maintaining the inhibition of enzyme activity, improving the therapeutic effect, and reducing the toxic and side effects caused by large blood concentration fluctuations.

The sustained release phase containing the matrix polymer for adjusting release rate can be prepared by sufficiently mixing the pharmaceutically active ingredient in an improved dissolution form, the matrix polymer for adjusting release rate, a diluent, and other excipients, and the like, compressing the same by the conventional methods well known to those skilled in the art (sustained release phase); the niraparib in an improved dissolution form according to the present invention is selected from a corresponding salt compound of niraparib free base, a co-grinding mixture made of niraparib and other matrix excipients, a niraparib nanocrystal or a niraparib solid dispersion, preferably a niraparib solid dispersion and a compound in a salt form, more preferably, a niraparib solid dispersion.

The sustained release phase comprises 100-900 parts by weight, preferably 150-700 parts by weight, more preferably 200-600 parts by weight of the above-mentioned the niraparib in an improved dissolution form, 10-300 parts by weight, preferably 30-150 parts by weight of the matrix polymer for adjusting release rate, 0-50 parts by weight of the diluent, and 0.2-30 parts by weight, preferably 1-30 parts by weight of other additives commonly used in tablets, and is prepared by sufficiently mixing each of the components, and then compressing the same by conventional methods well known to those skilled in the art.

The matrix polymer for adjusting release rate of the present invention is a sustained release matrix material well known to those skilled in the art, and may be one or a combination of two or more selected from the group consisting of polyoxyethylene, hydroxypropyl cellulose, hypromellose, methyl cellulose, hydroxyethyl cellulose, ethyl cellulose, sodium alginate, povidone, copovidone, acrylic resin, and carbomer, preferably hydroxypropyl cellulose, sodium alginate, hypromellose and carbomer; the diluent in the present invention is one or a combination of two or more selected from the following materials which are well known to those skilled in the art, such as microcrystalline cellulose, pregelatinized starch, sucrose, mannitol, sorbitol, sucrose, starch, sodium carboxymethyl starch; other common additives for tablet according to the present invention includes one or a combination of two or more of a lubricant, a colorant commonly used in solid preparations well known to those skilled in the art, the lubricant is one or a combination of two or more selected from the group consisting of magnesium stearate, stearic acid, sodium stearyl fumarate, talc, and colloidal silicon dioxide, and the colorant is one or a combination of two or more selected from the group consisting of iron oxide red, iron oxide yellow, iron oxide purple, iron oxide black, and titanium oxide.

The optional immediate release phase may comprise the above-described niraparib in an improved dissolution form, a disintegrant, an optional diluent, and other additives commonly used in tablets, or may comprise niraparib, a matrix polymer for solubilization, and other additives commonly used in tablets. It may be prepared by the following two preparation methods:

The first method comprises: sufficiently mixing a pharmaceutically active ingredient in an improved dissolution form, a disintegrant, a diluent, and other excipients, and then compressing the same by a conventional method well known to those skilled in the art (immediate release phase), wherein the pharmaceutically active ingredient in an improved dissolution form is selected from the group consisting of a grinding mixture, a nanocrystalline or solid dispersion of niraparib, preferably a niraparib solid dispersion, and the amount thereof may be 20-600 parts by weight, preferably 30-400 parts by weight, more preferably 50-250 parts by weight; the disintegrant is one or a combination of two or more selected from the group consisting of crospovidone, sodium carboxymethyl starch, low-substituted hydroxypropyl cellulose, cross-linked polyethylene pyrrolidone, croscarmellose sodium and other common pharmaceutical disintegrants, and the amount thereof may be 5-90 parts by weight, preferably 10-50 parts by weight; the diluent in the present invention is one or a combination of two or more selected from the following materials well known to those skilled in the art, such as microcrystalline cellulose, pregelatinized starch, sucrose, mannitol, sorbitol, sucrose, starch, sodium carboxymethyl starch, and the amount thereof may be 0-90 parts by weight, preferably 0-50 parts by weight; the other additives commonly used in tablets in the present invention comprises one or a combination of two or more of lubricants and colorants which are commonly used in solid preparations and well known to those skilled in the art, and the amount thereof may be 0.1-30 parts by weight, preferably 1-15 parts by weight, the lubricant is one or a combination of two or more selected from the group consisting of magnesium stearate, stearic acid, sodium stearyl fumarate, talc and colloidal silicon dioxide, and the colorant is one or a combination of two or more selected from the group consisting of iron oxide red, iron oxide yellow, iron oxide violet, iron oxide black, and titanium oxide.

The second method for preparing the optional immediate release phase comprises the following steps: dissolving the free base of the pharmaceutically active ingredient or a compound in a salt form thereof, a matrix polymer for solubilization and other excipient components, and then coating the resultant onto the sustained release phase, drying the same to form an immediate release coat film. The pharmaceutically active ingredient is niraparib, which may be used in an amount of 5-100 parts by weight, preferably 10-80 parts by weight, more preferably 20 to 60 parts by weight; the matrix polymer component for solubilization is one or a combination of two or more selected from the group consisting of povidone, copovidone, Soluplus, hypromellose phthalate (HPMCP), polyethylene glycol, poloxamer, hypromellose (HPMC) and other materials, which may be used in an amount of 5-300 parts by weight, preferably 10-200 parts by weight, more preferably 30-120 parts by weight; the other excipient ingredients include additives that are commonly used in immediate release tablets and well known to those skilled in the art, such as crospovidone, microcrystalline cellulose, sodium dodecyl sulfate and pharmaceutically acceptable surfactants, etc., and which may be used in an amount of 0.1-150 parts by weight, preferably 0.5-100 parts by weight.

The immediate and sustained double-effect matrix controlled release agent in the present invention comprises the sustained release phase carrier and/or the immediate release phase carrier; the pharmaceutically active ingredient released from the sustained release phase is contained in the sustained release phase carrier, and the drug released from the immediate release phase is contained in the immediate release phase carrier; the preparation with the immediate and sustained double-effect release behavior is characterized in that, based on the total amount of the pharmaceutically active ingredient in the preparation of the present invention, the pharmaceutically active ingredient in the immediate release phase accounts for 10-50 wt %, preferably 20-40 wt % of the total amount of the drug; and the sustained release phase contains 50-90 wt %, preferably 50-80 wt %, of the pharmaceutically active ingredient.

The niraparib controlled release preparation with the immediate and sustained double-effect release behavior of the present invention is characterized in that, for the pharmaceutically active ingredient in the immediate release phase, in accordance with the requirements specified in the drug release test, the Chinese Pharmacopoeia 2015, in the release medium that meets the sink condition, preferably more than 90 wt % of the pharmaceutically active ingredient dispensed into the immediate release phase is released within 2 hours, more preferably, more than 90 wt % of the pharmaceutically active ingredient dispensed into the immediate release phase is released within 1 hour; The release time for 90 wt % or more of the pharmaceutically active ingredient in the sustained release phase is preferably 10-16 hours, more preferably, releasing 90 wt % or more of the pharmaceutically active ingredient in 16 hours, and the release behavior of the pharmaceutically active ingredient in the sustained release phase is in accordance with the zero-order, first-order, Higuchi or the Ritger-Peppas release model, preferably a zero-order release model.

3. Sustained Release Pellet-Based Sustained and Controlled Release Tablets

The present invention provides a controlled release preparation consisting of a sustained release pellet and an optional immediate release matrix. The release behavior of the present invention can be realized by a tablet consisting of a sustained release pellet and an optional immediate release matrix.

The sustained release pellet-based sustained and controlled release tablet of niraparib in the present invention may be a sustained release pellet-based sustained release tablet and an immediate/sustained release pellet-based immediate and sustained double-effect tablet; in the immediate and sustained double-effect release tablet, the immediate release matrix constitutes an immediate release phase of immediate and sustained double-effect release tablet, and the sustained release pellet constitutes a sustained release phase of the immediate and sustained double-effect release tablet; based on the total weight of the pharmaceutical active ingredient, the active ingredient niraparib in the immediate release phase accounts for 10-40 wt % of the total active drug content in the entire immediate and sustained double-effect release tablet; the active ingredient niraparib in the sustained release pellet accounts for 60-90 wt % of the total active drug content in the entire immediate and sustained double-effect release tablet.

The tablet consisting of the sustained release pellet and an immediate release matrix in the present invention structurally comprises an immediate release matrix and a sustained release pellet; the immediate release matrix can be prepared by thoroughly mixing the pharmaceutically active ingredient in an improved dissolution form, a disintegrant, a non-limiting diluent and other excipients, compressing the same by conventional methods well known to those skilled in the art (immediate release phase). Wherein the pharmaceutically active ingredient in an improved dissolution form is selected from the group consisting of a compound of the niraparib in a salt form, a co-grinding mixture of a free base thereof, a niraparib nanocrystal or a solid dispersion, preferably a compound of the niraparib in a salt form and a niraparib solid dispersion, more preferably a niraparib solid dispersion. In an immediate release matrix comprising a pharmaceutically active ingredient in an improved dissolution form, the niraparib in an improved dissolution form may be used in an amount of 20-200 parts by weight, preferably 50-150 parts by weight; said disintegrant is one or a combination of two or more selected from the group consisting of crospovidone, sodium carboxymethyl starch, low-substituted hydroxypropyl cellulose, cross-linked polyethylene pyrrolidone, croscarmellose sodium and other common pharmaceutical disintegrants, and the amount thereof may be 5-200 parts by weight, preferably 10-100 parts by weight, more preferably 20-80 parts by weight; the diluent in the present invention is selected from the following materials well known to those skilled in the art, such as one or a combination of two or more selected from the group consisting of microcrystalline cellulose, pregelatinized starch, sucrose, mannitol, sorbitol, sucrose, starch, sodium carboxymethyl starch, and the amount thereof may be 0-200 parts by weight, preferably 10-150 parts by weight; the other excipients in the present invention comprises one or a combination of two or more of the lubricants and colorants commonly used in solid preparations well known to those skilled in the art, and the amount thereof may be 0.2-30 parts by weight, preferably 1-30 parts by weight, the lubricant is one or a combination of two or more selected from the group consisting of magnesium stearate, stearic acid, sodium stearyl fumarate, talc and colloidal silicon dioxide, and the colorant is one or a combination of two or more selected from the group consisting of iron oxide red, iron oxide yellow, iron oxide violet, iron oxide black, and titanium oxide.

The sustained release pellet may be prepared by: preparing pharmaceutically active ingredient or the pharmaceutically active ingredient in an improved dissolution form, a matrix for adjusting release rate, and optionally other excipients, and the like into sustained release pellets by a conventional method well known to those skilled in the art, such as wet granulation, extrusion spheronization, coating in a coating pan and/or fluidized bed granulation coating; e.g. prepared by dispersing or coating the pharmaceutically active ingredient and matrix polymer for solubilization in/onto the blank pellet core by means of a one-pot coating and loading drug in a coating pan to form a drug-loaded pellet core, and then coating a matrix sustained release coating film material for adjusting release rate on the drug-loaded pellet core to form the coated sustained release pellet. The blank pellet core in the present invention is one selected from the group consisting of a sucrose pellet core, a starch pellet core, a microcrystalline cellulose pellet core, a silica pellet core, and a hydroxypropyl cellulose pellet core; also, e.g., prepared by placing an active drug, a matrix polymer for solubilization and the sustained release matrix material for adjusting release rate or the like in a fluidized bed, blowing into air, mixing the drug and the excipients uniformly, and then spraying the binder thereto to granulate the same, here pelletizing, drying and coating are performed in one step.

The pharmaceutically active ingredient in the sustained release pellet of the present invention is niraparib; the matrix polymer component for solubilization is one or a combination of two or more selected from the group consisting of povidone, copovidone, Soluplus, hypromellose phthalate (HPMCP), polyethylene glycol, poloxamer, hypromellose (HPMC), polymethacrylate, hydroxypropyl cellulose, and other polymer excipients for solubilization; the sustained release matrix material for adjusting release rate in the sustained release pellet is one or a mixture of several materials selected from the common commercially available sustained release coating film materials, such as, shellac, cellulose acetate phthalate (CAP), acrylic resin (Eudragit), ethyl cellulose (EC), carbomer, polypropylene polysiloxane, cellulose acetate, cellulose propionate, cellulose acetate propionate, polyvinyl alcohol, polyvinylpyrrolidone (PVP), methyl cellulose, hydroxypropyl cellulose, hydroxypropyl methylcellulose (HPMC) and Eudragit, etc.; the other excipients in the sustained release pellet mainly comprises, but not limited to, a binder, a plasticizer, a porogen, etc.; wherein the binder is selected from the group consisting of polyethylene alcohol (PEG), stearic acid, glyceryl monostearate, etc., the plasticizer is selected from the group consisting of propylene glycol, glycerin, polyethylene glycol (PEG), triacetin, acetyl monoglyceride, phthalate, castor oil, etc., the porogen is selected from the group consisting of a hydrophilic liquid carriers (such as glycerin, PEG 200), saccharides (such as lactose, fructose, sucrose, mannose), surfactants (such as polysorbate 80, sodium dodecyl sulfate, etc.), polymers (such as povidone, hypromellose, etc.).

In one embodiment, the sustained release pellet comprises 100-500 parts by weight, preferably 200-400 parts by weight of a blank pellet core, 10-150 parts by weight, preferably 30-100 parts by weight of niraparib hydrochloride, 10-300 parts by weight of a matrix for adjusting release rate or a controlled release coating film material, 0-100 parts by weight of a binder, 0-12 parts by weight of a porogen, and 0-15 parts by weight of a plasticizer.

Finally, the sustained release pellets are directly compressed to prepare a sustained release preparation. According to the actual release requirements, the immediate release matrix and the sustained release pellets are uniformly mixed in a certain ratio, and then compressed into tablets by tablet press with a special stirring function, and then an immediate and sustained double-release preparation is prepared.

The sustained and controlled release capsule preparation, one of niraparib compositions in the present invention, may be selected from the group consisting of a pellet-based sustained and controlled release capsule and a microchip-based sustained and controlled release capsule; wherein the pellet-based sustained and controlled release capsule comprises a capsule containing a matrix-type sustained release pellet, a capsule containing a coated sustained release pellet, an immediate and sustained double-effect capsule containing an immediate release pellet and a matrix-type sustained release pellet, and an immediate and sustained double-effect capsule osmotic pump controlled release tablet containing an immediate release pellet and a coated sustained release pellet; and the microchip-based sustained and controlled release capsule comprises a capsule containing a matrix-type sustained release microchip and an immediate and sustained double-effect capsule containing an immediate microchip and a matrix-type sustained release microchip; the above sustained and controlled release capsules can realize the release behavior of the present invention by the means shown as below:

Wherein, FIG. 5 is a schematic view showing the structure of a capsule containing an immediate release and sustained release tablet, FIG. 6 is a schematic view showing the structure of a capsule containing an immediate release pellet and a matrix-type sustained release pellet according to an embodiment of the present invention, and FIG. 7 is a schematic view showing the structure of a capsule containing a sustained release pellet coated with an immediate release coat according to an embodiment of the present invention.

4. Microchip-Based Sustained and Controlled Release Capsules

The microchip-based sustained and controlled release capsule of the present invention is a controlled release capsule composed of a sustained release tablet or an immediate and sustained double-release capsule composed of a sustained release microchip and an immediate release microchip, and may include a capsule containing a matrix-type sustained release microchip, a capsule containing a matrix-type sustained release microchip having an immediate release coat and a capsule containing an immediate release microchip and a matrix-type sustained release microchip. In general, the tablets produced have small diameters, typically <5 mm, so as to be filled into hard capsules.

For the immediate and sustained double-effect release capsule, the immediate-release microchips constitute the immediate release phase, and the sustained release microchips constitute the sustained release phase. Based on the total weight of niraparib in the capsule, niraparib in the immediate release phase accounts for 10-40 wt %; and niraparib in the sustained release phase accounts for 60-90 wt %.

For the matrix-type sustained release tablet, the compositions, preparation method, materials and content and the like thereof are the same as those described for the sustained release phase in the matrix-type controlled release tablet in the above section 2, and their detailed descriptions are omitted here.

A matrix-type sustained release tablet containing an immediate release coat can be prepared by directly coating an immediate release matrix onto the surface of the above matrix-type sustained release tablet.

The immediate release tablet can be prepared by directly compressing the immediate release matrix.

For the immediate release matrix, the compositions, materials and contents thereof are the same as those described for the immediate release matrix in the above section 3, and their detailed descriptions are omitted here.

The sustained release capsule preparation may be prepared by encapsulating the matrix-type sustained release tablet, and the immediate and sustained double-release capsule may be prepared by uniformly mixing the immediate release tablet and the sustained release tablet in a certain ratio, and encapsulating the same, or encapsulating the matrix-type sustained release tablet containing the immediate release coat.

5. Pellet-Based Sustained and Controlled Release Capsules

The present invention provides a sustained and controlled release preparation consisting of a sustained release pellet and an optional immediate release pellet, and the release behavior of the present invention can be the realized by the capsule preparation consisting of the sustained release pellet and the optional immediate release pellet.

The pellet-based sustained and controlled release capsule of the present invention may be a sustained release pellet-based sustained release capsule, and an immediate release pellet and a sustained release pellet-based immediate and sustained double-effect capsule. For the immediate and sustained double-effect release capsule, the immediate release pellets constitute an immediate release phase, and the sustained release pellets constitute a sustained release phase. Based on the total weight of niraparib in the immediate and sustained double-effect release capsule, niraparib in the immediate release phase accounts for 10-40 wt %; and niraparib in the sustained release pellets accounts for 60-90 wt %.

For the coated sustained release pellets and the matrix-type sustained release pellets, the compositions, preparation method, materials and contents thereof are the same as those described for the sustained release pellets in the above section 3, and the detailed descriptions thereof are omitted here.

The sustained release pellets containing the immediate release coat can be prepared by directly coating the immediate release matrix on the surface of the above-mentioned matrix-type sustained release pellets or coated sustained release pellets.

The immediate release pellets can be prepared by dissolving the immediate release matrix, coating it onto a blank pellet core by a conventional coating method well known to those skilled in the art, or directly preparing the immediate release matrix into pellets.

For the immediate release matrix, the compositions, materials and contents thereof are the same as those described for the immediate release matrix in the above section 3, and the detailed descriptions thereof are omitted here.

The controlled release capsule can be prepared by encapsulating the sustained release pellets, and the immediate and sustained double-release capsule preparations can be prepared by weighing the above immediate release pellets and the sustained release pellets in a certain ratio, mixing them and then encapsulating the mixture, or the immediate and sustained double-release capsule preparations can be prepared by encapsulating the sustained release pellets containing the immediate release coat.

### EXAMPLES

The preparation and/or characterization results of typical compositions of the present invention is generally recorded in the following examples, all percentages are represented by weight, unless otherwise indicated. The present invention is described in details by the following examples, however, it should not be understood that these examples are intended to limit the scope of the present invention. In the following examples, various processes and methods that are not described in details are conventional methods well known in the art.

Test Animals: Beagle dogs, half male and half female, weighing 8-10 kg, MaXMaK Biotechnology Co., Ltd, Beijing. The test animals were adaptively fed for 14 days before the test day at the test site of the Experimental Animal Center, Shanghai Institute of Meteria Medica, Chinese Academy of Sciences.

Compression was performed on a single punch tablet press (TDP-1, Xulang Machinery Equipment Co., Ltd., Guangzhou).

The three-dimensional mixer: T2F model available from TURBULA.

The melt extruder: Pharma 11 model available from Thermo Fisher Scientific.

### Example 1 Immediate and Sustained Double-Effect Release Matrix Tablets

| Immediate release layer | Dosage (g) |
|---|---|
| niraparib | 20 |
| Soluplus | 60 |
| Colloidal silicon dioxide | 1 |
| crospovidone (PVPP XL) | 8 |
| magnesium stearate | 3 |

| Sustained release layer | Dosage (g) |
|---|---|
| niraparib | 80 |
| copovidone (PVP VA64) | 200 |
| Colloidal silicon dioxide | 2.5 |
| HPMC K15M | 75 |
| magnesium stearate | 3 |

Immediate release layer: a formula dosage of niraparib, the matrix excipient for solubilization (Soluplus) and the colloidal silicon dioxide were uniformly mixed, and then prepared into a solid dispersion by melt extrusion, the resultant was pulverized and sieved through a 60 mesh, and uniformly mixed with the disintegrant (crospovidone PVPP XL) and the lubricant (magnesium stearate) in a formula dosage to give a mixture to be compressed.

Sustained release layer: a formula dosage of niraparib and matrix excipient for solubilization (copovidone (PVP VA64)) and colloidal silicon dioxide were prepared into a solid dispersion by the above melt extrusion method, which was then uniformly mixed with a formula dosage of the sustained release matrix material for adjusting release rate (HPMC K15M (BASF, Germany)) and the lubricant (magnesium stearate) to give a mixture to be compressed.

Compression: an immediate and sustained double-effect release matrix tablet with appropriate hardness was prepared by direct compression method.

The release rate of the controlled release preparation was measured by using the apparatus II of the dissolution test method (Chinese Pharmacopoeia 2010 edition, Part II appendix XC), with the conditions as follows: temperature: 37° C., a release medium: buffers with different pH values, and the rotation speed: 75 rpm, according to the method, 6 mL of the solution was sampled at 0.25, 0.5, 0.75, 1, 2, 4, 6, 8, 10, 12 and 13 h and centrifuged, and the supernatant was used as a test solution to determine the release rate.

The release rate of the formula tablets was determined by measuring the absorbance at a wavelength of 240 nm according to the UV-visible spectrophotometry (Chinese Pharmacopoeia 2010 edition Part II, Appendix IV A).

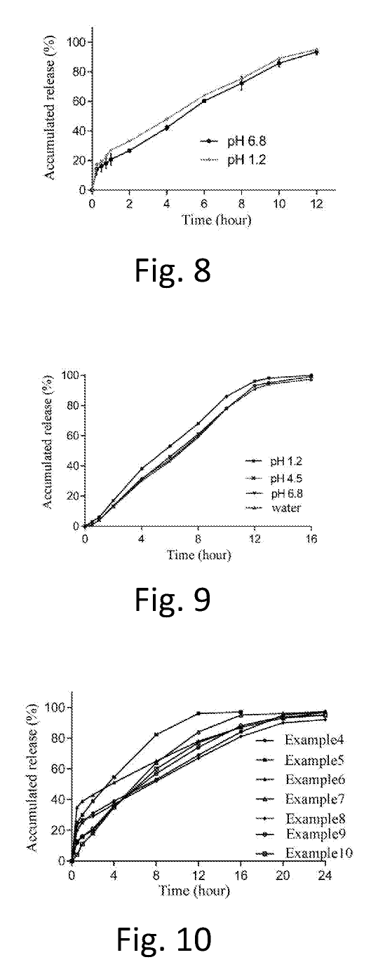

The release results are shown in FIG. 8. For the immediate and sustained double-effect matrix tablet, nearly 20% of the drug can be rapidly released within 30 min, and about 80% of the drug can be released in about 8 hours, and the remaining drug can be completely released at around 12-13 h. Thus it shows the following release behavior, after oral administration of niraparib, a portion of the drug can be rapidly absorbed to reach the desired blood concentration, then the drug is slowly absorbed by the slow release of drug to prevent the blood concentration peak value from rising too high and maintain the blood concentration required for effectively inhibiting PARP enzyme.

### Example 2 Immediate and Sustained Double-Effect Capsules Containing Immediate Release Pellet and Sustained Release Pellet (Immediate and Sustained Double-Release Capsule)

1 Sustained Release Pellet

I) Drug-Loaded Pellet Core

| Name | Dosage | |
|---|---|---|
| niraparib hydrochloride | 100 | g |
| microcrystalline cellulose pellet core | 400 | g |
| hydroxypropylcellulose (SSL) | 100 | g |
| 95% ethanol | 400 | ml |

II) Seal Coating

| Name | Dosage | |
|---|---|---|
| drug-loaded pellet core | 600 | g |
| povidone (K30) | 50 | g |
| 95% ethanol | 200 | ml |

III) Coating Sustained Release Coat

| Name | Dosage | |
|---|---|---|
| drug-loaded pellet core coated with a seal coating | 650 | g |
| Surelease (aqueous dispersion) | 200 | ml |
| water | 200 | ml |

2. Immediate Release Pellet

| Name | Dosage | |
|---|---|---|
| niraparib hydrochloride | 25 | g |
| copovidone (VA64) | 50 | g |
| microcrystalline cellulose pellet core | 100 | g |
| 95% ethanol | 300 | ml |

The preparation method was as follows:

Immediate release pellet: niraparib hydrochloride and copovidone (VA64) were dissolved or dispersed in a 95% ethanol solution to prepare a drug-loaded solution, which was sprayed onto a formula dosage of blank microcrystalline cellulose pellet core by a fluidized bed coating method to provide an immediate release pellet.

Sustained release pellet: I) Preparation of drug-loaded pellet core: a formula dosage of hydroxypropylcellulose (SSL) was dispersed in 95% ethanol solution to prepare a coating solution with a solid content of 10%, which was thoroughly stirred on a magnetic stirrer; and a formula dosage of niraparib was weighed, and dispersed in the above coating solution uniformly to provide a drug-loaded coating solution.

The microcrystalline cellulose pellet core was fed to the fluidized bed at the following operating parameters, air volume (100 m³/h) and inlet air temperature (30-54° C.), and the prepared drug-loaded coating solution was sprayed thereto for drug loading.

II) coating the seal coating: the seal coating film component, povidone (K30), was dissolved or dispersed in a 95% ethanol solution, which was sprayed onto the drug-loaded pellet core in step I) in a formula dosage by a fluidized bed coating method;

III) coating the sustained release coat: the aqueous dispersion of the sustained release coating solution, Surelease, was diluted by adding an appropriate amount of aqueous solution to give a Surelease coating solution with a solid content of 10-15 wt %, which was mixed uniformly to provide a sustained release coat film coating solution, which was then sprayed onto the drug-loaded pellet core obtained in step II) by a fluidized bed coating method to prepare a sustained release pellet.

Encapsulation: the above prepared sustained release pellets were encapsulated to give sustained release capsules; the above prepared immediate release pellets and sustained release pellets were mixed in different ratios, and encapsulated to obtain capsule preparations of the active drug niraparib with different immediate release/sustained release ratios. Generally, the amount of the active ingredient in the immediate release pellets was less than 40% of the total amount of the active pharmaceutical ingredient in the entire capsule.

Example 3 Osmotic Pump Controlled Release Tablet Preparations

| Ingredients of the drug-containing layer | Dosage | |
|---|---|---|
| niraparib | 5 | g |
| copovidone(VA64) | 12 | g |
| povidone(K90) | 2 | g |
| magnesium stearate | 0.3 | g |

| Ingredients of the boost layer | Dosage |
|---|---|
| sodium carboxymethyl starch | 8.4 g |
| hypromellose (K15M) | 1.7 g |
| Carbomer (971P) | 0.6 g |
| sodium chloride | 5.8 g |
| copovidone (VA64) | 3.6 g |
| iron oxide red | 0.2 g |
| magnesium stearate | 0.2 g |

A solid dispersion was prepared with niraparib and copovidone VA64 by solvent evaporation method, that is, niraparib and copovidone VA64 were simultaneously dissolved in an organic solvent of ethanol/acetone (40:60), and the organic solvent was evaporated under reduced pressure. The resultant was dried and pulverized, uniformly mixed with povidone K90 and magnesium stearate in a formula dosage, sieved, and uniformly mixed by a three-dimensional mixer to obtain a controlled release drug-containing layer composition to be compressed.

The boost layer excipient was accurately weighed, sieved and mixed by a three-dimensional mixer (25 rpm, 30 min) to obtain a boost layer composition. An osmotic pump double-layer tablet core is given by a direct compression method.

The pressed tablet core was coated with 4% of cellulose acetate solution in acetone, and the coating film gained the weight by 10% and a conventional controlled release osmotic pump tablet was prepared.

The release rate of the controlled release osmotic pump tablet was determined by the method for determining the release rate of the controlled release preparation in Example 1.

The release results in different pH release media are shown in FIG. 9. The results shows that the double-layer osmotic pump controlled release tablets are basically not affected by pH. The active ingredient niraparib can basically be released at a constant rate, with less than 10% release in 1 hour, about 50% release in 6 hours, and 80% or more release in 12 hours. The total release duration can last for 14 hours.

Example 4 Immediate and Sustained Double-Release Double-Layer Osmotic Pump Controlled Release Tablet Preparations

| Ingredients of the tablet core of the drug-containing | | |
|---|---|---|
| layer | Dosage | |
| niraparib | 75 | g |
| copovidone(VA64) | 200 | g |
| povidone(K90) | 20 | g |
| sodium dodecyl sulfate | 5 | g |
| magnesium stearate | 3 | g |

| Ingredients of the tablet core of the boost layer | Dosage | |
|---|---|---|
| sodium carboxymethyl starch | 77 | g |
| hypromellose (K15M) | 17 | g |
| Carbomer(971P) | 6 | g |
| sodium chloride | 55 | g |
| copovidone (VA64) | 33 | g |
| iron oxide black | 1 | g |
| magnesium stearate | 2 | g |

Niraparib and copovidone were sieved through a 60 mesh sieve for 3 times, and then mixed by a three-dimensional mixer at 30 rpm for 25 min. The mixture was slowly added to the preheated melt extruder to collect the transparent extrudate, which was pulverized and sieved through a 60 mesh sieve to obtain the niraparib solid dispersion. The obtained niraparib solid dispersion and other excipients other than magnesium stearate were sieved through a 60 mesh sieve in a formula dosage and mixed by a three-dimensional mixer at 30 rpm for 25 min, and magnesium stearate was added thereto and mixed therewith for another 5 min to obtain a drug-containing layer composition, which was to be compressed.

The boost layer excipient was accurately weighed and sieved through a 60 mesh sieve and mixed by a three-dimensional mixer at 30 rpm for 30 min to obtain a boost layer composition.

The osmotic pump double-layer tablet core comprising the drug-containing layer and the boost layer was obtained by pressing the above-described drug-containing layer composition and the boost layer composition by a direct compression method.

A controlled release coat layer was coated on the pressed tablet core with a 3% cellulose acetate-0.2% PEG4000 solution, and the coating film gained the weight by 10%, and a double-layer osmotic pump controlled release tablet was obtained.

The niraparib solid dispersion was dissolved in the acetone solution, and the resultant was coated onto the double-layer osmotic pump tablet with the ratio of the drug contained in the immediate release layer to that in the sustained release layer being 25:75, to give an immediate and sustained double-release double-layer osmotic pump tablet in which the active ingredient in the immediate release layer accounted for 25 wt % and the active ingredient in the sustained release layer accounted for 75 wt %.

The release rate of the immediate and sustained double-release double-layer osmotic pump controlled release tablet was determined at 37° C. by using the apparatus II of the dissolution test method (Chinese Pharmacopoeia, 2010 edition, Part II, Appendix X C), and 900 ml of sodium acetate buffer (80 mM, pH 4.0) was used as the release medium, the rotation speed was 50 rpm, according to the method, 6 mL of the solution was sampled at 0.5, 1, 2, 4, 8, 12, 16, 20, 24 h, and centrifuged, and the supernatant was used as a test solution to determine the release rate.

The release rate of the formula tablets was determined by measuring the absorbance at a wavelength of 240 nm according to the UV-visible spectrophotometry (Chinese Pharmacopoeia 2010 edition Part II, Appendix IV A).

The release results are shown in FIG. 10. The results show that the immediate and sustained double-release double-layer osmotic pump controlled release tablet can release the drug in the immediate release layer within 2 hours, and 80% or more of the drug in the sustained release layer can basically be released in 16 hours at a constant rate, and the release duration can last for 20 hours.

Example 5 Sustained Release Matrix Coating Tablets Containing an Immediate Release Coat Layer

1 Preparation of Sustained Release Tablet Core

| Name | Amount | |
|---|---|---|
| niraparib | 7 | g |
| poloxamer 188 | 21 | g |
| sodium alginate | 6 | g |
| magnesium stearate | 0.2 | g |

2 Immediate Release Coat

| Name | Amount | |
|---|---|---|
| niraparib | 3 | g |
| poloxamer 188 | 9 | g |
| talc | 1 | g |
| polyethylene glycol 4000 | 2 | g |
| 95% ethanol | 90 | g |
| water | 10 | g |

The preparation method was as follows:

Preparation of sustained release tablet core in the sustained release matrix coating tablets: a formula dosage of niraparib and poloxamer 188 were sieved through a 60 mesh sieve and mixed in a three-dimensional mixer at 30 rpm for 25 min, and then slowly added to the preheated melt extruder, and the extrudate was collected, pulverized and sieved through a 60 mesh sieve to obtain a niraparib solid dispersion. The niraparib solid dispersion prepared as above was mixed uniformly with the matrix polymer for adjusting dissolution rate (sodium alginate), and then added with a lubricant (magnesium stearate) and mixed, and the resulting mixture was compressed by a direct compression method to obtain a sustained release tablet core with suitable hardness.

Coating the immediate release coat: an immediate release coat solution was prepared according to the recipe of the immediate release coat, and the above sustained release tablet core was coated with the immediate release coat in a high-efficiency coating pan; finally, the resultant was dried at 45° C. for 12 hours to remove the remaining organic solvent and moisture, and the sustained release matrix coated tablets were obtained.

The release rate was determined in the same manner as in Example 4, sodium acetate buffer (80 mM, pH 4.0) was used as the release medium, and the release results are shown in FIG. 10.

Example 6 Sustained Release Pellet-Based Sustained and Controlled Release Tablets

1 Sustained Release Pellet

I) Drug-Loaded Pellet Core

| Name | Amount | |
|---|---|---|
| niraparib | 100 | g |
| sucrose blank pellet core | 400 | g |
| copovidone (VA64) | 200 | g |
| 95% ethanol | 400 | ml |

II) Coating a Seal Coating

| Name | Amount | |
|---|---|---|
| drug-loaded pellet core | 700 | g |
| hypromellose (E5) | 50 | g |
| 95% ethanol | 200 | ml |

III) Coating a Sustained Release Coat

| Name | Amount | |
|---|---|---|
| drug-loaded pellet core coated with a seal coating | 750 | g |
| Eudragit NE30D | 150 | g |
| talc | 5 | g |
| PEG 4000 | 15 | g |
| water | 750 | ml |

The preparation method was as follows:

Drug-loaded pellet core: niraparib and VA64 were dissolved or dispersed in a 95% ethanol solution to prepare a drug-loaded solution, which was sprayed onto a formula dosage of sucrose blank pellet core by a fluidized bed coating method to give a drug-loaded pellet core.

Sustained Release Pellets:

The component of the seal coating was dissolved or dispersed in a 95% ethanol solution, and sprayed onto a formula dosage of the drug-loaded pellet core by a fluidized bed coating method to obtain a drug-loaded pellet core coated with a seal coating.

The aqueous dispersion of the sustained release coating solution was diluted with an appropriate amount of the aqueous solution, mixed, and the mixture was used as a sustained release coating film coating solution, which was sprayed onto the drug-loaded pellets core coated with the seal coating by a fluidized bed coating method to give sustained release pellets.

Sustained and controlled release tablets: the microcrystalline cellulose was added with ethanol to form granules, which were uniformly mixed with the sustained release pellets, and then added with silica or magnesium stearate, and the resultant was uniformly mixed and compressed.

Immediate and sustained double-release tablets: the above-prepared immediate release pellets (drug-loaded pellet core) and sustained release pellets were thoroughly mixed in the formula dosage, and then silica or magnesium stearate were added, and the resultant was mixed uniformly and compressed. The release rate was determined in the same manner as in Example 4, sodium acetate buffer (80 mM, pH 4.0) was used as the release medium, and the release results are shown in FIG. 10.

### Example 7 Sustained Release Pellet-Based Sustained and Controlled Release Tablets

154 g of niraparib p-toluenesulfonate hydrate, 140 g of microcrystalline cellulose and 100 g of lactose were weighed and sieved through a 80 mesh sieve, and then transferred to a wet granulator with the parameters adjusted, 1 wt % of hypromellose E15 aqueous solution was added as the adhesive for the preparation of the soft material, which was extruded and spheronized to give the niraparib-containing pellets, wherein the extrusion screen had a pore size of 0.5 mm, with an extrusion speed of 20 r/min and a spheronization speed of 1000 r/min, after dried in a fluidized bed at 40° C., the resultant was sieved through a 30-40 mesh sieve to give the drug-containing pellets. The screened niraparib pellets were placed in a fluidized bed and a coating solution was prepared to coat, then the niraparib sustained release pellets were prepared. The composition of the coating solution comprises 14.5% of acrylic resin, 5% of plasticizer (triethyl citrate), 10.5% of anti-adhesive (talc), the remaining of water. 25 g of niraparib sustained release pellets, 5 g of drug-containing pellets, 12 g of microcrystalline cellulose, 16 g of lactose, 12 g of 5 wt % of pvpK30 solution were weighed and granulated through a 18 mesh sieve, dried in oven at 40° C., sieved through a 18 mesh sieve, added with 0.6 g of stearic acid, mixed and compressed.

The release rate was determined in the same manner as in Example 4, sodium acetate buffer (80 mM, pH 4.0) was used as the release medium, and the release results are shown in FIG. 10.

### Example 8 Microchip-Based Sustained and Controlled Release Capsules

Sustained Release Microchip

| Name | Amount | |
|---|---|---|
| niraparib | 10 | g |
| copovidone (PVP VA64) | 22 | g |
| polyoxyethylene | 5 | g |
| ethyl cellulose | 2 | g |
| magnesium stearate | 0.3 | g |

Immediate Release Microchip

| Name | Amount | |
|---|---|---|
| niraparib | 10 | g |
| copovidone (PVP VA64) | 22 | g |
| crospovidone | 3 | g |
| magnesium stearate | 0.3 | g |

Sustained release microchip: niraparib and copovidone VA64 were sieved through a 60 mesh sieve for 3 times, and ground in a ball mill to an average particle diameter of less than 30 μm to obtain a niraparib co-grinding mixture. The co-grinding mixture was sieved through a 60 mesh sieve together with a matrix polymer for adjusting release rate, polyoxyethylene and ethyl cellulose in a formula dosage and mixed in a three-dimensional mixer at 30 rpm for 25 min, and then added with magnesium stearate and mixed therewith for 5 min, and compressed to microchips with a diameter of 4 mm.

Immediate release microchip: niraparib and copovidone VA64 were sieved through a 60 mesh sieve for 3 times, and ground in a ball mill to an average particle size of less than 30 μm to obtain a niraparib co-grinding mixture. The co-grinding mixture was sieved through a 60 mesh sieve with crospovidone in a formula dosage and mixed in a three-dimensional mixer at 30 rpm for 25 min, and then added with magnesium stearate and mixed therewith for 5 min, and compressed to microchips with a diameter of 4 mm.

Encapsulation: The sustained release microchips prepared as above were encapsulated to prepare sustained release capsules.

The immediate release microchips and the sustained release microchips prepared as above were thoroughly mixed in the formula dosage, and then encapsulated to prepare the immediate and sustained double-release capsules.

The release rate was determined in the same manner as in Example 4, sodium acetate buffer (80 mM, pH 4.0) was used as the release medium, and the release results are shown in FIG. 10.

### Example 9 Microchip-Based Sustained and Controlled Release Capsules

Sustained Release Microchip

| Name | Amount | |
|---|---|---|
| niraparib | 15 | g |
| 2-hydroxypropyl-β-cyclodextrin | 30 | g |
| Carbomer 934 | 7 | g |
| sodium stearyl fumarate | 0.3 | g |

Sustained Release Microchip

| Name | Amount | |
|---|---|---|
| niraparib | 15 | g |
| 2-hydroxypropyl-β-cyclodextrin | 30 | g |
| lactose | 8 | g |
| croscarmellose sodium | 2 | g |
| sodium stearyl fumarate | 0.3 | g |

Sustained release microchip: Niraparib and 2-hydroxypropyl-β-cyclodextrin were sieved through a 60 mesh sieve for 3 times, after adding 100 ml of water to shear it at high-speed, a crude suspension was obtained, which was cycled homogenized with a high-pressure homogenizer to an average particle diameter of less than 1000 nm, and the nanocrystal solution was lyophilized in a lyophilizer to remove moisture. The nanocrystalline powder was sieved through a 60 mesh sieve, the resultant was sieved through a 60 mesh sieve in a formula dosage together with the matrix polymer for adjusting dissolution rate, carbomer 934 and mixed in a three-dimensional mixer at 30 rpm for 25 min, and the resultant was added with sodium stearyl fumarate and mixed for 5 min, and then compressed to microchips with a diameter of 3 mm.

Immediate release microchip: niraparib and 2-hydroxypropyl-β-cyclodextrin were sieved through a 60 mesh sieve for 3 times. After adding 100 ml of water to shear it at high-speed, a crude suspension was obtained, which was cycled homogenized with a high-pressure homogenizer to an average particle diameter of less than 1000 nm, and the nanocrystal solution was lyophilized in a lyophilizer to remove moisture. The nanocrystalline powder was sieved through a 60 mesh sieve, and sieved through a 60 mesh sieve together with lactose, croscarmellose sodium in the formula dosage and mixed in a three-dimensional mixer at 30 rpm for 25 min, and the resultant was added with sodium stearyl fumarate and mixed for 5 min, and then compressed to microchips with a diameter of 3 mm.

Encapsulation: The sustained release microchips prepared as above were encapsulated to prepare sustained release capsules.

The immediate release microchips and the sustained release microchips prepared as above were thoroughly mixed in the formula dosage, and then encapsulated to prepare immediate and sustained double-release capsules. The release rate was determined in the same manner as in Example 4, sodium acetate buffer (80 mM, pH 4.0) was used as the release medium, and the release results are shown in FIG. 10.

### Example 10 Single Layer Osmotic Pump Controlled Release Tablets

| Ingredients of the tablet core | Amount | |
|---|---|---|
| niraparib | 50 | g |
| Hypromellose E5 | 150 | |
| povidone(K90) | 52 | g |
| povidone (K30) | 25 | g |
| sodium chloride | 90 | g |
| sodium dodecyl sulfate | 5 | g |
| magnesium stearate | 4 | g |

The formula dosage of niraparib and hypromellose E5 were sieved through a 60 mesh sieve and mixed in a three-dimensional mixer at 30 rpm for 25 min, and then slowly added to the preheated melt extruder. The extrudate was collected, pulverized and sieved through a 60 mesh sieve to obtain a niraparib solid dispersion. The niraparib solid dispersion prepared as above was sieved through a 60 mesh sieve with other excipients other than magnesium stearate and uniformly mixed in a three-dimensional mixer, and the resultant was further mixed with magnesium stearate for 5 min to obtain a drug-containing layer composition to be compressed.

The single layer osmotic pump tablet core was obtained by pressing the above-described drug-containing layer composition by a direct compression method. The pressed tablet core was coated with a 4% cellulose acetate-0.2% PEG4000 solution to form a controlled release coat layer, and the coating film gained the weight by 5% and a single-layer osmotic pump controlled release tablet was obtained. The release rate was determined in the same manner as in Example 4, sodium acetate buffer (80 mM, pH 4.0) was used as the release medium, and the release results are shown in FIG. 10.

### Comparative Example 1

Immediate release capsule 1 (self-made) was prepared by uniformly mixing 20 wt % of niraparib hydrochloride, 43 wt % of microcrystalline cellulose, 32 wt % of lactose, 2 wt % of colloidal silicon dioxide, 1 wt % of magnesium stearate and 2 wt % of sodium dodecyl sulfate, and directly loading the mixture into 0# gelatin hard capsules. The dissolution rate was determined by using the apparatus I of the dissolution test (Chinese Pharmacopoeia 2010 edition Part II Appendix X C), with the conditions as below: temperature: 37° C., the release medium: 900 mL of aqueous solution of hydrochloric acid (pH 1.2), rotation speed: 75 rpm. According to the method, 6 mL of the solution was sampled at predetermined time points, centrifuged, and the supernatant was taken as the test solution to determine the release rate.

The dissolution rate of the capsules was determined by measuring the absorbance at a wavelength of 240 nm according to the UV-visible spectrophotometry (Chinese Pharmacopoeia 2015 edition Part II, Appendix IV A).

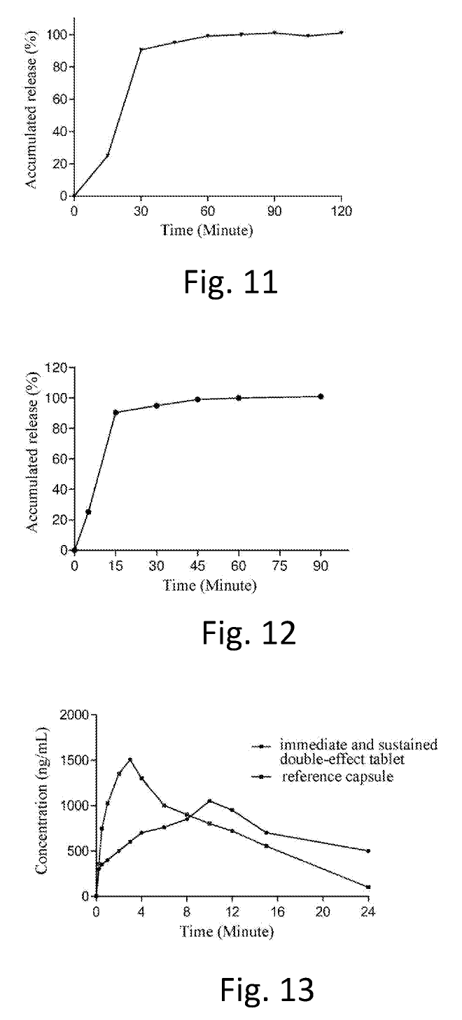

The release results are shown in FIG. 11. 85% or more of the active ingredient niraparib in the immediate release capsule is released in about 30 min, and almost all is released within 1 hour.

### Comparative Example 2

Immediate release capsule 2 (self-made) was prepared by uniformly mixing 50 wt % of niraparib p-toluenesulfonate hydrate, 49 wt % of lactose monohydrate, and 1 wt % of magnesium stearate, and directly loading the mixture into 0# gelatin hard capsule. The dissolution rate was determined by using the apparatus I of the dissolution test method (Chinese Pharmacopoeia 2015 edition Part II Appendix XC), with the conditions as below: temperature: 37° C., release medium: 900 mL of sodium acetate buffer (80 mM, pH 4.0), rotation speed: 50 rpm. According to the method, 6 mL of the solution was sampled at predetermined time points, centrifuged, and the supernatant was taken as the test solution to determine the release rate.

The dissolution rate of the capsules was determined by measuring the absorbance at a wavelength of 240 nm according to the UV-visible spectrophotometry (Chinese Pharmacopoeia 2010 edition Part II, Appendix IV A).

The release results are shown in FIG. 12. 80% or more of the active ingredient niraparib in the immediate release capsule is released in about 45 min, and almost all is released within 1 hour.

### Experimental Example 1

Niraparib immediate release capsules (Comparative Example 1) and the immediate and sustained double-effect controlled release tablets (Example 1) were separately administered to full-fed beagle dogs (n=3) with 25 mL of water, respectively. After administration, blood was taken at predetermined time points, and the blood sample was centrifuged at 4000 rpm for 10 min at 4° C., and the upper layer plasma was taken for blood concentration detection by LC-MS. The results are shown in FIG. 13. Compared with the Cₘₐₓ (1503.4 ng/mL) of the capsule preparation, the Cₘₐₓ of the immediate and sustained double-effect controlled release tablets is reduced to 1050.1 ng/mL, that is, reduced by about 30%; the AUC_{0-24 h} change <10%; As seen from the drug-time curve in FIG. 13, compared with the immediate release capsule, the case with the immediate and sustained double-effect matrix tablets can reach a certain blood concentration in a short time by the initial immediate release means, and then realize a slow rise of the drug blood concentration by the later sustained release means, so that the blood concentration is prevented from rising sharply, and the blood concentration required for effectively inhibiting the PARP enzyme is maintained for a long time, thereby exhibiting better enzyme inhibition effect and the anti-tumor effect, and providing larger room for drug dose escalation and optimal drug efficacy.

### Experimental Example 2

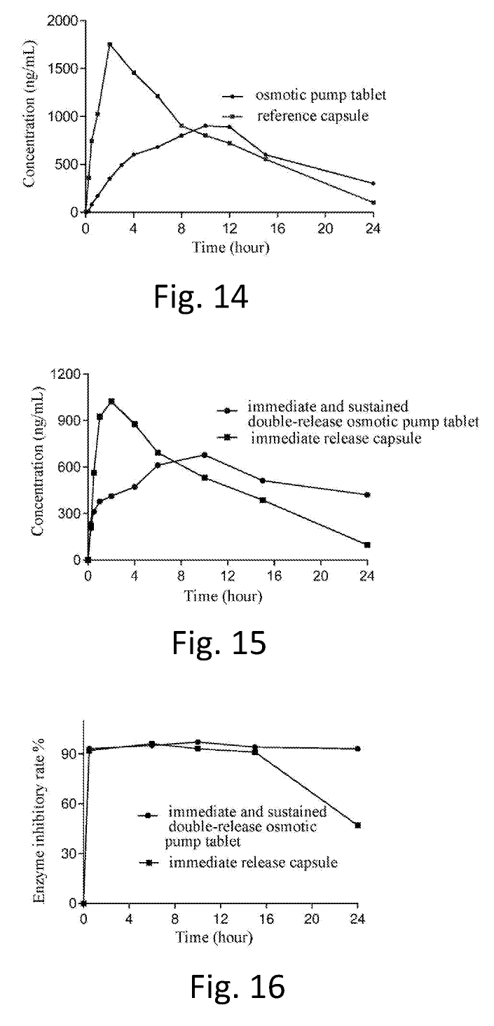

Niraparib immediate release capsules (Comparative Example 1) and the double-layer osmotic pump controlled release tablets (Example 3) were separately administered to full-fed beagle dogs (n=3) with 25 mL of water, respectively. After administration, blood was taken at predetermined time points, and the blood sample was centrifuged at 4000 rpm for 10 min at 4° C. to obtain the upper plasma for blood concentration detection by LC-MS. The results are shown in FIG. 14. Compared with the Cₘₐₓ (1754.0 ng/mL) of the capsule preparation, the Cₘₐₓ of the double-layer osmotic pump tablet is reduced to 903.2 ng/mL, that is, reduced by about 49%; the AUC_{0-24 h} change <30%; As seen from the drug-time curve in FIG. 14, compared with the immediate release capsule, the double-layer osmotic pump tablet realizes the slow drug absorption, realizes the slow rise of drug blood concentration, and the peak time and half-life of the blood concentration are prolonged to prevent blood concentration from rising sharply, so it is expected to exhibit better enzyme inhibition effects and anti-tumor effects, while providing larger room for drug dose escalation and optimal drug efficacy.

### Experimental Example 3

Niraparib immediate release capsules (Comparative Example 2) and the immediate and sustained double-release double-layer osmotic pump controlled release tablets (Example 4) were administered to full-fed beagle dogs (n=3) with 25 mL of water, respectively. After administration, blood was taken at predetermined time points, and the blood sample was centrifuged at 4000 rpm for 10 min at 4° C., and the upper plasma was taken for blood concentration detection by LC-MS. The results are shown in FIG. 15. PBMC were extracted from whole blood at 0 h, 0.5 h, 6 h, 10 h, 15 h, and 24 h, and PARP inhibition was assayed by HT PARP in vivo Pharmacodynamic Assay II kit, Trevigen. The results are shown in FIG. 16.

Compared with the Cₘₐₓ (1138.7 ng/mL) of the capsule preparation, the Cₘₐₓ of the double-layer osmotic pump tablet is reduced to 678.0 ng/mL, that is, reduced by about 40%; the AUC_{0-24 h} change <30%; the immediate release capsule has an enzyme inhibition rate of lower than 50% at 10 hours, and the immediate and sustained double-release double-layer osmotic pump tablets has an enzyme inhibition rate of higher than 90% at 10 h, and the period that the enzyme inhibition level is higher than IC90 can last for 10 h. As seen from drug-time curve in FIG. 15 and the enzyme inhibition rate graph in FIG. 16, compared with the immediate release capsule, the double-layer osmotic pump tablet realizes the slow absorption of the drug, realizes the slow rise of the drug blood concentration, the peak time and half-life of the blood concentration are prolonged to prevent the blood concentration from rising sharply, so that the enzyme inhibition rate is maintained for a long time. It is expected to exhibit better enzyme inhibition effects and anti-tumor effects, while providing larger room for drug dose escalation and optimal drug efficacy.

1. A niraparib sustained and controlled release pharmaceutical composition, comprising a niraparib in an improved dissolution form and a matrix polymer for adjusting release rate;
the niraparib sustained and controlled release pharmaceutical composition has a steady-state blood concentration trough value C_{min,ss} of 0.5-4 μM; a steady-state blood concentration peak value C_{max,ss} of 0.8-6 μM.

2. The niraparib sustained and controlled release pharmaceutical composition according to claim 1, wherein the niraparib pharmaceutical composition has a steady-state blood concentration trough value C_{min,ss} of 1-3 μM; a steady-state blood concentration peak value C_{max,ss} of 2-5 μM, and the steady-state blood concentration peak/trough ratio is preferably less than 2, more preferably less than 1.5.

3. The niraparib sustained and controlled release pharmaceutical composition according to claim 1, wherein the niraparib pharmaceutical composition has a controlled release behavior, the release behavior and the release amount thereof can be controlled in a release medium under the sink condition within a predetermined period of time, when determining the release behavior in a buffer solution with a pH of 1.2-7.8 at 37° C. using the apparatus II of the dissolution test method in the Chinese Pharmacopoeia, the release amount of niraparib in 1 hour is less than 50%, preferably 30%, more preferably 10-25% of the total amount of niraparib; the release amount of niraparib in 16 hours is greater than 80%, more preferably 90% of the total amount of niraparib.

4. The niraparib sustained and controlled release pharmaceutical composition according to claim 1, wherein
the niraparib in an improved dissolution form comprises: a corresponding salt compound of niraparib free base, a niraparib co-grinding mixture, niraparib nanocrystal, and a niraparib solid dispersion,
preferably, the matrix polymer for adjusting release rate is selected from the group consisting of cellulose derivatives, starch or derivatives thereof, alginate, acrylic or methacrylic acid derivatives, polyethylene oxide, gums and carbohydrate-based polymer, preferably, one or a combination of two or more selected from the group consisting of polyoxyethylene, hydroxypropyl cellulose, hypromellose, methylcellulose, hydroxyethylcellulose, ethylcellulose, cellulose acetate, sodium alginate, povidone, copovidone, acrylic resin and carbomer, preferably, one or a combination of two or more selected from the group consisting of polyoxyethylene, hydroxypropyl cellulose, sodium alginate, hypromellose, and carbomer;
preferably, the compound of the niraparib in a salt form is selected from the group consisting of a hydrochloride, a phosphate, a besylate, a maleate, a sulfate, and a d-camphorate;
preferably, the niraparib co-grinding mixture consists of active drug niraparib, a matrix polymer for solubilization, and other additives, and is prepared by co-grinding the above ingredients; in the co-grinding mixture, based on the total weight of the co-grinding composition, the weight percentage of niraparib is 5 to 60 wt %, preferably 20 to 40 wt %, and the weight percentage of the matrix polymer for solubilization is 40 to 95 wt %, preferably 40 to 80 wt %, the weight percentage of the other additives is 0-15 wt %, preferably 0.2-10 wt %;
preferably, the niraparib nanocrystal consists of active drug niraparib, a matrix polymer for solubilization, and/or optionally other additives, and is obtained by preparing the above ingredients into nano-sized particles by high pressure homogenization method or coprecipitation method; in the niraparib nanocrystal, based on the total weight of the niraparib nanocrystals, the weight percentage of niraparib is 10-100 wt %, preferably 20-50 wt %; the weight percentage of the matrix polymer for solubilization is 0-75 wt %, preferably 0-65 wt %, and the weight percentage of the other additives is 0-10 wt %, preferably 0-5 wt %; the particle size of the nanocrystal is preferably 50-1000 nm;
preferably, the solid dispersion consists of active drug niraparib, a matrix polymer for solubilization, and optionally other additives, and is prepared by solvent evaporation method or melt extrusion method, in the solid dispersion, based on the total weight of the solid dispersion, the weight percentage of niraparib is 5-50 wt %, preferably 10-40 wt %, more preferably 20-40 wt %, and the weight percentage of the matrix polymer for solubilization is 45-95 wt %, preferably 50-80 wt %, and the weight percentage of the other additives is 0-12 wt %, preferably 0-10 wt %;
preferably, the matrix polymer for solubilization comprises one or a combination of two or more selected from the group consisting of povidone, copovidone, polyoxyethylene, Soluplus, hypromellose phthalate (HPMCP), hydroxypropyl cellulose acetate succinate, polyethylene glycol, poloxamer, polymethacrylic acid, polyethyl acrylate, 2-hydroxypropyl-β-cyclodextrin, hypromellose (HPMC), polymethacrylate, hydroxypropyl cellulose, cellulose acetate phthalate (CAP), and other pharmaceutically acceptable conventional polymeric excipients for solubilization;
preferably, the other additives are selected from common pharmaceutical plasticizers and/or lubricants, etc., preferably, the plasticizer may be one or a combination of two or more selected from PEG 4000, phthalates, small molecular surfactant (such as Cremphor RH40 and polyoxyethylene (40) stearate), and other common pharmaceutical plasticizers, and the lubricants may be one or a combination of two or more selected from the common lubricants, such as colloidal silicon dioxide, magnesium stearate and the like.

5. The niraparib sustained and controlled release pharmaceutical composition according to claim 1, comprising 50-900 parts by weight, preferably 80-700 parts by weight, more preferably 120-600 parts by weight of the niraparib in an improved dissolution form; and 10-300 parts by weight, preferably 20-250 parts by weight, more preferably 50-180 parts by weight of the matrix polymer for adjusting release rate;
preferably, the niraparib oral sustained and controlled release pharmaceutical composition comprises:
50-700 parts by weight of the compound of niraparib in a salt form, and 10-300 parts by weight of the matrix polymer for adjusting release rate; or
50-700 parts by weight of the niraparib co-grinding mixture, and 10-200 parts by weight of the matrix polymer for adjusting release rate; or
50-800 parts by weight of niraparib nanocrystal, and 0-250 parts by weight of the matrix polymer for adjusting release rate; or
50-900 parts by weight of the niraparib solid dispersion, and 20 to 300 parts by weight of the matrix polymer for adjusting release rate.

6. The niraparib sustained and controlled release pharmaceutical composition according to claim 1, which is a sustained and controlled release preparation containing a single sustained release phase or an immediate and sustained double-effect release preparation containing both an immediate release phase and a sustained release phase, wherein
preferably,
the sustained release phase is a controlled release composition containing a pharmaceutically active ingredient, which is selected from a controlled release composition in a controlled release tablet, a controlled release pellet or a tablet, a controlled release composition in a tablet or a pellet core, a controlled release layer composition incorporated into a double-layer tablet and any combinations thereof;
the immediate release phase is an immediate release composition containing a pharmaceutically active ingredient, which is selected from an immediate release composition in an immediate release tablet, an immediate release pellet or a tablet, an immediate release coat layer coating on a controlled release tablet or a pellet core, and an immediate release layer composition in a double-layer controlled release tablet, and any combinations thereof.

7. The niraparib sustained and controlled release pharmaceutical composition according to claim 6, wherein in the immediate and sustained double-effect controlled release preparation, the pharmaceutically active ingredient in the immediate release phase accounts for 10-50 wt %, preferably 20-40 wt % of the total amount of the pharmaceutically active ingredient; the pharmaceutically active ingredient in the sustained release phase accounts for 50-90 wt %, preferably 60-80 wt % of the total amount of the pharmaceutically active ingredient.

8. The niraparib sustained and controlled release pharmaceutical composition according to claim 1, which is a tablet or capsule, preferably selected from the group consisting of an osmotic pump controlled release tablet, an osmotic pump immediate and sustained double-release tablet, a matrix-type sustained release tablet, a matrix-type immediate and sustained double-effect double-layer tablet, a matrix-type immediate and sustained double-effect coated tablet, a sustained release pellets-based sustained release tablet, a sustained release pellet and immediate release pellet-based immediate and sustained double effect table, a capsule containing a matrix-type sustained release pellet, a capsule containing a coated sustained release pellet, a capsule containing a sustained release pellet coated with an immediate release coat, an immediate and sustained double-release capsule containing an immediate release pellet and a matrix-type sustained release pellet, an immediate and sustained double-release capsule containing an immediate release pellet and a coated sustained release pellet, a capsule containing a matrix-type sustained release microchip, a capsule containing a matrix-type sustained release microchip coated with an immediate release coat and a capsule containing an immediate release microchip and a matrix-type sustained release microchip.

9. Use of the niraparib sustained and controlled release pharmaceutical composition of claim 1 in the preparation of a drug for preventing or treating a tumor, in particular a tumor selected from the group consisting of ovarian cancer, breast cancer, gastric cancer, lung cancer, blood cancer, pancreatic cancer, glioblastoma, epithelial ovarian cancer and metastatic brain cancer and the like.

10. The use according to claim 9, wherein the recommended total dose/day of the niraparib sustained and controlled release pharmaceutical composition is 100-800 mg/day, preferably 200-500 mg/day, the amount of the pharmaceutically active ingredient niraparib contained in a unit preparation (such a single preparation or capsule) is not particularly limited and may be selected as needed, for example, it may contain 20 to 400 mg, preferably 50 to 400 mg of the pharmaceutically active ingredient.
